(19) United States
(12) Patent Application Publication
(10) Pub. No.: US 2026/0354734 A1
(43) Pub. Date: Nov. 12, 2026

(54) A METHOD FOR ELECTROENCEPHALOGRAM (EEG) CAPTURE AND RELATED ELECTRONIC DEVICE

(71) Applicant: BrainCapture ApS, Kongens Lyngby (DK)

(72) Inventors: Tue LEHN-SCHIØLER, Kongens Lyngby (DK); Radu Calin GATEJ, Kongens Lyngby (DK); Paul LOOMIS, Kongens Lyngby (DK)

(73) Assignee: BrainCapture ApS, Kongens Lyngby (DK)

(21) Appl. No.: 19/142,344

(22) PCT Filed: Dec. 20, 2023
(86) PCT No.: PCT/EP2023/086923

§ 371 Date: Jun. 23, 2025

(30) Foreign Application Priority Data
Dec. 23, 2022 (DK) PA202270650

(51) Int. Cl. A61B 5/372; A61B 5/00

(52) U.S. Cl. CPC A61B 5/372; A61B 5/7207

(57) ABSTRACT

An electronic device and related methods are disclosed, the electronic device comprising an interface and one or more processors, wherein the electronic device is configured to perform a first cycle. To perform the first cycle comprises to obtain first sensor data indicative of electrical activity of a brain of a subject; to determine, using a disturbance model, first disturbance data including a first disturbance parameter based on the first sensor data; to determine whether the first disturbance data satisfies one or more first disturbance criteria; in accordance with a determination that a first primary disturbance criterion of the one or more first disturbance criteria is satisfied, to provide a first primary instruction via the interface.

The present disclosure pertains to the field of monitoring electrical activity of a brain and relates to a method of monitoring electrical activity of a brain and related electronic device.

### BACKGROUND

An electroencephalogram (EEG) is a method of measuring the electrical activity of a brain. A particular use of the EEG method is of epilepsy diagnosis. Data obtained during an EEG test may include epileptiform spikes which may be attributed to the patient having epilepsy. However, lateral eye movement by the patient during the test may cause false positive epileptiform spikes and muscle movements may mask the epileptiform spikes. Typically, the EEG method is performed under supervision of a trained medical professional who may adjust recording conditions to reduce recording artifacts and ensure a high quality result. The quality of EEG result is important such that an accurate diagnosis can be provided based on the result.

### SUMMARY

The access to trained medical professionals can be limited and there is a need for an electronic device and a method that can simplify and/or improve the monitoring and capture of electrical activity of a brain such as for diagnosis of brain disease. Accordingly, there is a need for an electronic device and a method for obtaining accurate EEG data with reduced requirements for trained medical professionals.

Disclosed is an electronic device comprising an interface and one or more processors. The electronic device is configured to perform a first cycle. In one or more example electronic devices, to perform the first cycle comprises to obtain first sensor data indicative of electrical activity of a brain of a subject. In one or more example electronic devices, to perform the first cycle comprises to determine, using a disturbance model, first disturbance data including a first disturbance parameter based on the first sensor data. In one or more example electronic devices, to perform the first cycle comprises to determine whether there is a disturbance, such as whether the first disturbance data satisfies one or more first disturbance criteria. In one or more example electronic devices, to perform the first cycle comprises, in accordance with a determination that there is a disturbance, e.g. that a first primary disturbance criterion of the one or more first disturbance criteria is satisfied, to provide a first primary instruction via the interface.

Further, a method of monitoring electrical activity of a brain is disclosed, the method performed by an electronic device comprising an interface and one or more processors. The method comprises performing a first cycle. In one or more example methods, performing the first cycle comprises obtaining first sensor data indicative of electrical activity of a brain of a subject. In one or more example methods, performing the first cycle comprises determining, using a disturbance model, first disturbance data including a first disturbance parameter based on the first sensor data. In one or more example methods, performing the first cycle comprises determining whether there is a disturbance, such as whether the first disturbance data satisfies one or more first disturbance criteria. In one or more example methods, performing the first cycle comprises, in accordance with a determination that there is a disturbance, such as that a first primary disturbance criterion of the one or more first disturbance criteria is satisfied, providing a first primary instruction via the interface.

It is an advantage of the present disclosure that a user is informed about and guided on the EEG capture by dynamically providing instructions, which in turn leads to improved EEG capture and quality of EEG data obtained.

### BRIEF DESCRIPTION OF THE DRAWINGS

The above and other features and advantages of the present disclosure will become readily apparent to those skilled in the art by the following detailed description of exemplary embodiments thereof with reference to the attached drawings, in which:

FIG. 1A is a diagram illustrating schematically an example system for monitoring electrical activity of a brain according to this disclosure,

FIG. 1B is a flow chart illustrating an exemplary method, performed by an electronic device, of monitoring electrical activity of a brain according to this disclosure,

FIGS. 2A-2E are a flow-chart illustrating an exemplary method, performed by an electronic device, of monitoring electrical activity of a brain according to this disclosure,

FIG. 3 is a block diagram illustrating an exemplary electronic device according to this disclosure, and

FIGS. 4A-4E show different user interfaces of an electronic device.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The present disclosure relates to methods and devices for improving capture of EEG signals.

An electronic device is disclosed. The electronic device comprises an interface and one or more processors.

The electronic device is configured to perform a first cycle. The first cycle may be referred to as a first EEG capture cycle. To perform the first cycle comprises to obtain first sensor data indicative of electrical activity of a brain of a subject. To perform the first cycle comprises to determine, using a disturbance model, first disturbance data including a first disturbance parameter based on the first sensor data. The electronic device is for example configured to determine, using the disturbance model, first disturbance data, including a first disturbance parameter based on the first sensor data, using processor circuitry. In some examples, the first disturbance data can be seen as information indicative of an artifact, e.g., a disturbance. In some examples, the electronic device is configured to determine first disturbance data, based on first sensor data, associated with an action performed by the subject. An artifact can for example be seen in the first sensor data as a spike, peak etc. In some examples, an artifact can be seen as noise in the first sensor data. In some examples, an action performed by the subject can produce an artifact in the first sensor data. In some examples, an action can be seen as a movement performed by the subject, e.g., eye blinking, lateral eye movement, chewing, eye closing, and/or jaw clenching. In some examples, artifacts in the first sensor data can be generated by a first type of muscle movements, a second type of muscle movements, sweating, electrode pop and/or line noise. A first type of muscle movements may be muscle movements associated with facial expressions and/or a second type of muscle movements may be shivering. In some examples, the first disturbance parameter comprises a value, such as a number, associated with an artifact based of the first cycle. In some examples, the first disturbance parameter comprises a value, e.g., a number, associated with an artifact based on first sensor data. For example, the first disturbance parameter comprises a value, e.g., a number, associated with an action performed by the subject. In other words, the first disturbance parameter is for example indicative of an artifact associated with an action performed by the subject. In some examples, the electronic device is configured to determine the first disturbance parameter, based on the first sensor data, comprising a value indicative of the number and/or frequency of actions performed by the subject. For example, the first disturbance parameter may comprise information indicative of the number of eye blinks, lateral eye movements, chews, eye closings, and/or jaw clenches based on the first sensor data. In some examples, the first disturbance parameter comprises a value indicative of the intensity, such as magnitude, of the artifact associated with the action performed by the subject. In one or more example electronic devices, determining first disturbance data comprises determining the first disturbance parameter over a first time window. In one or more example electronic devices, the first time window is in the range from 10 seconds to 1 minute. The first time window is for example in the range from 20 seconds to 40 seconds. The first time window is for example in the range from 0.5 seconds to 30 seconds. For example, the first disturbance parameter may comprise values indicative of first artifacts determined within the first time window.

To perform the first cycle comprises to determine whether the first disturbance data satisfies one or more first disturbance criteria. To perform the first cycle comprises to, in accordance with a determination that a first primary disturbance criterion of the one or more first disturbance criteria is satisfied, provide a first primary instruction via the interface. In some examples, the electronic device is configured to repeat the first cycle one or more times. The first cycle can for example be seen as a loop. In some examples, the electronic device is configured to not repeat the first cycle. In some examples, the electronic device is configured such that whether the first cycle repeats, is determined by the processor circuitry. In some examples, the electronic device is configured such that whether the first cycle repeats is determined based on input, such as user input, via an interface of the electronic device. In other words, the subject may select, using the electronic device, whether to repeat the first cycle.

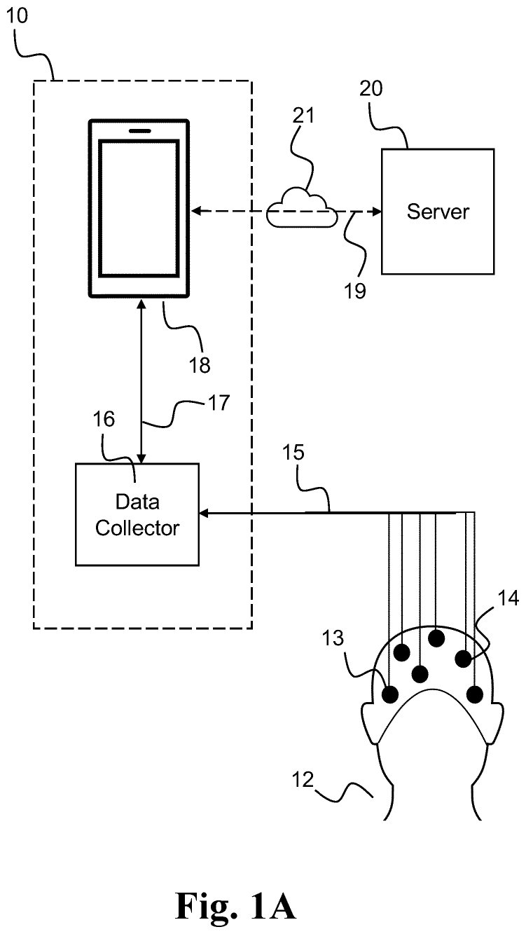

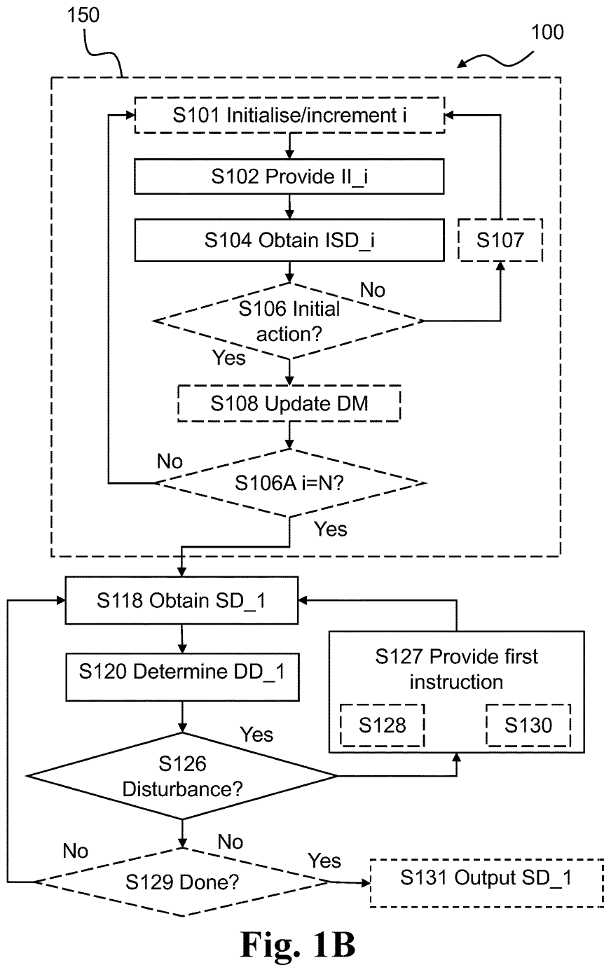

In one or more example electronic devices, the one or more first disturbance criteria comprises the first primary disturbance criterion and/or the first secondary disturbance criterion. Each of the one or more first disturbance criteria is for example associated with a first instruction. In some examples, the first primary disturbance criterion is associated with the frequency of artifacts. For example, the first primary disturbance criterion can be associated with the number of blinks, number of lateral eye movements, number of chews, number of times eyes closed, and/or number of times jaw clenched per unit time. For example, the first primary disturbance criterion is satisfied if the electronic device determines that the frequency of eye blinks within a specific time window, such as the first time window, is higher than a specified value, e.g., in the range from 5 to 10 blinks per 60 seconds. For example, the one or more first disturbance criteria can be seen as threshold values. For example, the primary disturbance criterion may comprise a threshold value greater than a normal human action frequency, e.g., blinks per second. In some examples, the first primary instruction is provided, such as to the subject, via an interface of the electronic device, such as the electronic device 10 of FIG. 1A. In one or more examples, the subject can be seen as a user, e.g., the user of the electronic device. In some examples, the user is a person different to the subject. The first primary instruction for example comprises one or more of an image, a video message, a text message, and an audio message. The first primary instruction is for example provided via a display of an electronic device. The first primary instruction is for example provided via a speakerphone of an electronic device. The first primary instruction may comprise a request for the subject to modify actions, such as to reduce actions, e.g., to reduce the frequency of artifacts. For example, the first primary instruction comprises a message instructing the subject to reduce frequency of actions, e.g., to blink less. In some examples, the first primary instruction comprises information indicative of the first disturbance data. For example, the first primary instruction may comprise the message, “Eye blinking has been determined 10 times in the last 30 seconds. Please reduce your eye blinking”. In some examples, the electronic device is configured to provide the first primary instruction in real time, such as while the electronic device obtains the first sensor data. In some examples, the first disturbance data comprises information indicative of more than one artifact, e.g., lateral eye movement and eye blinks. In some examples, the electronic device is configured to provide an instruction associated with more than one artifact, such as the primary artifact and the secondary artifact. For example, the first disturbance data may comprise a first primary disturbance parameter associated with lateral eye movements and a first secondary disturbance parameter associated with eye blinks. For example, if the first disturbance data comprising a first primary disturbance parameter associated with lateral eye movements satisfies the first primary disturbance criterion and a first secondary disturbance parameter associated with eye blinks satisfies the first secondary disturbance criteria, a first primary instruction associated with lateral eye movements and a first secondary instruction associated with eye blinks may be combined and provided as one message, e.g., “close eyes and relax”. In some examples, the first primary instruction is displayed on a light board using a set of lamps and/or light emitting diodes. In some examples, the first primary instruction is colour coded.

In some examples, the first primary disturbance criterion is associated with the intensity, e.g., magnitude of an artifact (such as a disturbance). In some examples, the electronic device is configured such that the first primary disturbance criterion is satisfied when the first disturbance data exceeds a specified value, such as a number. In other words, if a spike and/or peak exceeds a specified value the first primary disturbance criterion can be seen as being satisfied. The first primary instruction may comprise a request for the subject to modify actions, such as to reduce actions, e.g., to reduce the magnitude of artifacts. For example, the first primary instruction may comprise the message, “Excessive jaw clenching has been determined. Please reduce your jaw clenching/relax your jaw”. In some examples, poorly connected electrodes of the EEG cap may result in artifacts in the first sensor data. For example, the electronic device is configured to determine a specific electrode that may be improperly connected, e.g., based on sensor data. In some examples, the electronic device is configured to provide a first instruction indicative of adjusting the EEG cap and/or the electrodes, such as reconnecting a specific electrode and/or verifying good connection of a specific electrode.

In one or more example electronic devices, the electronic device is configured to, in accordance with a determination that a first secondary disturbance criterion of the one or more first disturbance criteria is satisfied, provide a first secondary instruction via the interface. In some examples, the first secondary disturbance criterion is associated with the frequency of artifacts. For example, the first secondary disturbance criterion can be associated with the number of blinks, number of lateral eye movements, number of chews, number of times eyes closed, and/or number of times jaw clenched per unit time. For example, the first secondary disturbance criterion is satisfied if the electronic device determines that the frequency of eye blinks within a specific time window, such as the first time window, is higher than a specified value, e.g.,. in the range from 5 to 10 blinks per 60 seconds. In some examples, the first secondary instruction is provided, such as to the subject, via an interface of the electronic device, such as the electronic device 10 of FIG. 1A. The first secondary instruction for example comprises an image, a video message, a text message and/or an audio message. The first secondary instruction is for example provided via a display of an electronic device. The first secondary instruction is for example provided via a speakerphone of an electronic device. The first secondary instruction may comprise a request for the subject to modify actions, such as to reduce actions (e.g., to reduce the frequency of artifacts). For example, the first secondary instruction comprises a message instructing the subject to reduce frequency of actions, e.g., to blink less. In some examples, the first secondary instruction comprises information indicative of the first disturbance data. For example, the first secondary instruction may comprise the message, “Eye blinking has been determined 10 times in the last 30 seconds. Please reduce your eye blinking”. In some examples, the electronic device is configured to provide the first secondary instruction in real time, optionally while the electronic device obtains the first sensor data. In some examples, the first disturbance data comprises information indicative of more than one artifact, e.g., lateral eye movement and eye blinks. In some examples, the electronic device is configured to provide an instruction associated with more than one artifact (such as the primary artifact and the secondary artifact). In some examples, the first secondary instruction is displayed on a light board using a set of lamps and/or light emitting diodes. In some examples, the first secondary instruction is colour coded.

In some examples, the first secondary disturbance criterion is associated with the intensity, e.g., magnitude, of an artifact (such as a disturbance). In some examples, the electronic device is configured such that the first secondary disturbance criterion is satisfied when the first disturbance data exceeds a specified value (such as a number). In other words, if a spike and/or peak exceeds a specified value the first secondary disturbance criterion can be seen as being satisfied. The first secondary instruction may comprise a request for the subject to modify actions, such as to reduce actions, e.g., to reduce the magnitude of artifacts. For example, the first secondary instruction may comprise the message, “Excessive jaw clenching has been determined. Please reduce your jaw clenching/relax your jaw”.

In some examples, the first sensor data is obtained via electrical sensors, e.g., electrodes, of an EEG cap (such as the EEG cap 14 shown in FIG. 1). For example, the EEG cap can be configured to obtain, via the electrodes, information indicative of the electrical activity in the brain. In some examples, the first sensor data can be seen as information indicative of the electrical activity of the brain of the subject. For example, the EEG cap is configured to obtain information indicative of the Delta frequency, Theta frequency, Alpha frequency and/or Beta frequency. In some examples, the EEG cap is configured to obtain first sensor data in the frequency range of 0.1 Hz to 60 Hz. In some examples, the EEG cap is configured to obtain first sensor data in the frequency range of 0.5 Hz to 32 Hz. In some examples, the first sensor data is indicative of the impedance, such as resistance and/or capacitance, of the electrodes. For example, the electronic device may be configured to provide information indicative of the impedance, e.g., indicative of the quality of the first sensor data of one or more electrodes of the EEG cap. In some examples, the electrodes of the EEG cap are wet electrodes, e.g., electrodes with an electrolyte gel to form a path between skin and electrode. In some examples, the electrodes of the EEG cap can be seen as dry electrodes. In some examples, due to the lack of electrolyte gel used for dry electrodes, the electronic device may obtain first sensor data indicative of a higher impedance than for first sensor data obtained via wet electrodes. In some examples, low quality sensor data can be seen as sensor data with a high frequency and/or magnitude of artifacts. In some examples, high quality sensor data can be seen as sensor data with a low frequency and/or magnitude of artifacts. In some examples, the first sensor data comprises epileptiform spikes.

In some examples, the subject is a person. In some examples, the electronic device is configured to monitor the electrical activity of a brain of the subject (such as a person).

For example, obtaining first sensor data indicative of electrical activity of a brain of a subject comprises obtaining first sensor data indicative of electrical activity of a brain of a person. In some examples, the subject my not be a person, e.g., the subject may be an animal.

In some examples, the disturbance model can be configured to determine artifacts based on the sensor data. In one or more example electronic devices, the disturbance model comprises machine learning and/or artificial intelligence models. For example, the disturbance model comprises machine learning algorithms. The machine learning model is for example trained on a set of data, e.g., sensor data, where artifacts have been pre-labelled, e.g., by trained medical professionals. For example, updating the disturbance model comprises updating machine learning algorithms of the disturbance model. In one or more example electronic devices, the machine learning algorithms are trained using artificial data generated by utilizing General Adversarial Networks (GAD). In some examples, the machine learning model is trained using binary classifiers individually for each artifact such as to enable the machine learning model to determine more than one artifact at the same time stamp in the data. In some examples, the disturbance model comprises Neural Networks, support vector machines, logistic regression and/or binary classification algorithms. In some examples, the disturbance model comprises decision trees, random forest and/or nearest neighbour approaches. In some examples, the disturbance parameter comprises a threshold signal processing algorithm, unsupervised machine learning classification and/or a Gaussian mixture model. In some examples, the disturbance model comprises a rule-based model. In some examples, updating the disturbance model comprises updating a rule-based model. For example, updating a rule-based model may comprise updating threshold values, e.g., one or more first and/or second disturbance criteria. In some examples, the electronic device is configured to determine, such as using the disturbance model, the cause of the artifact.

In some examples, artifacts may be caused by line noise, such as noise generated by proximal electric equipment lines. The line noise may cause an artifact (such as increased energy in the 50 Hz and/or 60 Hz bands) in the first sensor data associated with a frequency of 50 Hz and/or 60 Hz. In one or more example electronic devices, the disturbance model is configured to, using a notch filter, remove and/or reduce the line noise in the first sensor data. In some examples, the electronic device can adjust for the higher impedance, e.g., such as by filtering out high impedance associated with first sensor data obtained via dry electrodes. For example, the sensor data comprises electrode pops. Electrode pops can for example be caused by pressure and/or pulls on the electrode, poor electrode application (such as a weak contact patch between an electrode and the skin of the subject), dry electrodes (such as a dry contact patch between the electrode and the skin of the subject) and/or a dirty electrode (e.g., dirt, grit and/or dust between the electrode and the skin of the subject).

Performing the first cycle may comprise, e.g. in accordance with a determination that the first disturbance data does not satisfy any first disturbance criterion and/or after providing the first closing instruction, outputting, such as storing in memory and/or transmitting e.g. to server device, the first sensor data.

In one or more example electronic devices, the electronic device is configured to, after performing the first cycle, perform a second cycle. In one or more example electronic devices, to perform the second cycle comprises to obtain second sensor data indicative of electrical activity of the brain of the subject. In one or more example electronic devices, to perform the second cycle comprises to determine, using the disturbance model, second disturbance data including a second disturbance parameter based on the second sensor data. The electronic device is for example configured to determine, using the disturbance model, second disturbance data, including a second disturbance parameter based on the second sensor data, using processor circuitry. In some examples, the second disturbance data can be seen as information indicative of an artifact, e.g., a disturbance. In some examples, the electronic device is configured to determine second disturbance data, based on second sensor data, associated with an action performed by the subject. An artifact can for example be seen in the second sensor data as a spike, peak etc. In some examples, an artifact can be seen as noise in the second sensor data. In some examples, an action performed by the subject can produce an artifact in the second sensor data. In some examples, artifacts in the second sensor data can be generated by voluntary muscle movements, e.g., muscle movements associated with facial expressions, involuntary muscle movements (such as shivering), sweating, electrode pop and/or line noise. In some examples, the second disturbance parameter comprises a value, e.g., a number associated with an artifact based on second sensor data. For example, the second disturbance parameter comprises a value, e.g., a number, associated with an action performed by the subject. In other words, the second disturbance parameter is for example indicative of an artifact associated with an action performed by the subject. In some examples, the electronic device is configured to determine the second disturbance parameter, based on the second sensor data, comprising a value indicative of the number and/or frequency of actions performed by the subject. For example, the second disturbance parameter may comprise information indicative of the number of eye blinks, lateral eye movements, chews, eye closings, and/or jaw clenches based on the second sensor data. In some examples, the second disturbance parameter comprises a value indicative of the intensity (such as magnitude) of the artifact associated with the action performed by the subject. In one or more example electronic devices, to determine second disturbance data comprises to determine a second disturbance parameter over a second time window. In one or more example devices, the second time window is in the range from 10 seconds to 1 minute. The second time window is for example in the range from 20 seconds to 40 seconds. The second time window is for example in the range from 0.5 seconds to 30 seconds. For example, the second disturbance parameter may comprise values indicative of second artifacts determined within the second time window.

In one or more example electronic devices, to perform the second cycle comprises to determine whether the second disturbance data satisfies one or more second disturbance criteria. In one or more example electronic devices, to perform the second cycle comprises, in accordance with a determination that a second primary disturbance criterion of the one or more second disturbance criteria is satisfied, to provide a second primary instruction via the interface. In some examples, the electronic device is configured to repeat the second cycle one or more times. The second cycle can for example be seen as a loop. In some examples, the electronic device is configured to not repeat the second cycle. In some examples, the electronic device is configured such that whether the second cycle repeats, is determined by the processor circuitry. In some examples, the electronic device is configured such that whether the second cycle repeats is determined, based on input (such as user input) via an interface of the electronic device. In other words, the subject can select, using the electronic device, whether to repeat the second cycle.

In one or more example electronic devices, the one or more second disturbance criteria comprises the second primary disturbance criterion and/or the second secondary disturbance criterion. Each of the one or more second disturbance criteria is for example associated with a second instruction. In some examples, the second primary disturbance criterion is associated with the frequency of artifacts. For example, the second primary disturbance criterion can be associated with the number of blinks, number of lateral eye movements, number of chews, number of times eyes closed, and/or number of times jaw clenched per unit time. For example, the second primary disturbance criterion is satisfied if the electronic device determines that the frequency of eye blinks within a specific time window (such as the second time window) is higher than a specified value, e.g., in the range from 5 to 10 blinks per 60 seconds. For example, the one or more second disturbance criteria can be seen as threshold values. In some examples, the second primary instruction is provided, such as to the subject, via an interface of the electronic device, such as the electronic device 10 of FIG. 1A. The second primary instruction for example comprises an image, a video message, a text message and/or an audio message. The second primary instruction is for example provided via a display of an electronic device. The second primary instruction is for example provided via a speakerphone of an electronic device. The second primary instruction may comprise a request for the subject to modify actions, such as to reduce actions, e.g., to reduce the frequency of artifacts. For example, the second primary instruction comprises a message instructing the subject to reduce frequency of actions, e.g., to blink less. In some examples, the second primary instruction comprises information indicative of the second disturbance data. For example, the second primary instruction may comprise the message, “Eye blinking has been determined too many times in the last 30 seconds. Please reduce your eye blinking”. In some examples, the electronic device is configured to provide the second primary instruction in real time, such as while the electronic device obtains the second sensor data.

In some examples, the second disturbance data comprises information indicative of more than one artifact, e.g., lateral eye movement and eye blinks. In some examples, the electronic device is configured to provide one instruction associated with more than one artifact. For example, the second disturbance data may comprise a second primary disturbance parameter associated with lateral eye movements and a second secondary disturbance parameter associated with eye blinks. For example, if the second disturbance data comprising a second primary disturbance parameter associated with lateral eye movements satisfies the second primary disturbance criterion a second secondary disturbance parameter associated with eye blinks satisfies the second secondary disturbance criteria, a second primary instruction associated with lateral eye movements and a second secondary instruction associated with eye blinks may be combined and provided as one message, e.g., “close eyes and relax”. In some examples, the second primary instruction is displayed on a light board using a set of lamps and/or light emitting diodes. In some examples, the second primary instruction is colour coded.

In some examples, the second primary disturbance criterion is associated with the intensity, e.g., magnitude of an artifact, such as a disturbance. In some examples, the electronic device is configured such that the second primary disturbance criterion is satisfied when the second disturbance data exceeds a specified value (such as a number). In other words, if a spike and/or peak exceeds a specified value the second primary disturbance criterion is can be seen as being satisfied. The second primary instruction may comprise a request for the subject to modify actions, such as to reduce actions, e.g., reduce the magnitude of artifacts. For example, the second primary instruction may comprise the message, “Excessive jaw clenching has been determined. Please reduce your jaw clenching/relax your jaw”.

In one or more example electronic devices, the electronic device is configured to, in accordance with a determination that a second secondary disturbance criterion of the one or more second disturbance criteria is satisfied, provide a second secondary instruction via the interface. In some examples, the second secondary disturbance criterion is associated with the frequency of artifacts. For example, the second secondary disturbance criterion can be associated with the number of blinks, number of lateral eye movements, number of chews, number of times eyes closed, and/or number of times jaw clenched per unit time. For example, the second secondary disturbance criterion is satisfied if the electronic device determines that the frequency of eye blinks within a specific time window (such as the second time window) is higher than a specified value e.g., in the range from 5 to 10 blinks per 60 seconds. In some examples, the second secondary instruction is provided (such as to the subject) via an interface of the electronic device (such as the electronic device 10 of FIG. 1A). The second secondary instruction for example comprises an image, a video message, a text message and/or an audio message. The second secondary instruction is for example provided via a display of an electronic device. The second secondary instruction is for example provided via a speakerphone of an electronic device. The second secondary instruction may comprise a request for the subject to modify actions, such as to reduce actions, e.g., to reduce the frequency of artifacts. For example, the second secondary instruction comprises a message instructing the subject to reduce frequency of actions, e.g., to blink less. In some examples, the second secondary instruction comprises information indicative of the second disturbance data. For example, the second secondary instruction may comprise the message, “Eye blinking has been determined too many times in the last 30 seconds. Please reduce your eye blinking”. In some examples, the electronic device is configured to provide the second secondary instruction in real time, such as while the electronic device obtains the second sensor data. In some examples, the second secondary instruction is displayed on a light board using a set of lamps and/or light emitting diodes. In some examples, the second secondary instruction is colour coded.

In some examples, the second secondary disturbance criterion is associated with the intensity, e.g., magnitude of an artifact, such as a disturbance. In some examples, the electronic device is configured such that the second secondary disturbance criterion is satisfied when the second disturbance data exceeds a specified value (such as a number). In other words, if a spike and/or peak exceeds a specified value the second secondary disturbance criterion is can be seen as being satisfied. The second secondary instruction may comprise a request for the subject to modify actions, such as to reduce actions, e.g., to reduce the magnitude of artifacts. For example, the second secondary instruction may comprise the message, “Excessive jaw clenching has been determined. Please reduce your jaw clenching/relax your jaw”. In some examples, poorly connected electrodes of the EEG cap may result in artifacts in the second sensor data. In some examples, the electronic device is configured to provide a second instruction indicative of adjusting the EEG cap and/or the electrodes (such as reconnecting a specific electrode and/or verifying good connection of a specific electrode).

In some examples, the second sensor data is obtained via electrical sensors, e.g., electrodes, of an EEG cap. In some examples, the second sensor data can be seen as information indicative of the electrical activity of the brain of the subject. In some examples, the EEG cap is configured to obtain second sensor data in the frequency range of 0.1 Hz to 60 Hz. In some examples, the EEG cap is configured to obtain second sensor data in the frequency range of 0.5 Hz to 32 Hz. In some examples, the first sensor data is indicative of the impedance of the electrodes. For example, the electronic device may be configured to provide information indicative of the impedance, e.g., indicative of the quality of the first sensor data of one or more electrodes of the EEG cap. In some examples, due to the lack of electrolyte gel used for dry electrodes, the electronic device may obtain second sensor data indicative of a higher impedance than for second sensor data obtained via wet electrodes. In some examples, the second sensor data comprises epileptiform spikes.

Performing the second cycle may comprise, e.g. in accordance with a determination that the second disturbance data does not satisfy any second disturbance criterion and/or after providing the second closing instruction, outputting, such as storing in memory and/or transmitting e.g. to server device, the second sensor data.

In one or more example electronic devices, the electronic device is configured to perform a first initial cycle. In some examples, the first initial cycle is performed prior to the second initial cycle, first cycle and/or second cycle. In some examples, the first initial cycle can be seen as a calibration cycle. In one or more example electronic devices, to perform the first initial cycle comprises to provide a first initial instruction indicative of a first action via the interface. The first initial instruction is for example provided (such as to the subject) via the interface. The first initial instruction for example comprises an image, a video message, a text message and/or an audio message. The first initial instruction is for example provided via a display of an electronic device. The first initial instruction is for example provided via a speakerphone of an electronic device. In some examples, the first initial instruction may instruct the subject to create one or more artifacts in the first initial sensor data, e.g., by blinking eyes. For example, the first initial instruction may comprise the message “Please blink once every second for the next 10 seconds”. In some examples, the electronic device (such as the electronic device 18 of FIG. 1A) is configured to request subject input indicative of the start of the first initial cycle. For example, the electronic device may be configured to start the first initial cycle in accordance with a user input requesting the start of the first initial cycle being received, e.g., the user pushes a “start first initial cycle” button provided by the interface of the electronic device”. In one or more example electronic devices, to perform the first initial cycle comprises to obtain first initial sensor data indicative of electrical activity of the brain of the subject. In some examples, the first action can be seen as an action associated with the first initial cycle. The first action for example comprises eye blinking, lateral eye movement, chewing, eye closing, jaw clenching, closed eyes for a time period, open eyes for a time period, talking and/or head movement performed by the subject. In one or more example electronic devices, to perform the first initial cycle comprises to determine whether the first initial sensor data is indicative of the first action. In some examples, the first initial sensor data is obtained via electrical sensors, e.g., electrodes, associated with an EEG cap (such as the EEG cap 14 shown in FIG. 1A). In some examples, the first sensor data can be seen as information indicative of the electrical activity of the brain of the subject. In one or more example electronic devices, to perform the first initial cycle comprises to, e.g. in accordance with a determination that the first initial sensor data is indicative of the first action, update the disturbance model based on the first initial sensor data. In some examples, updating the disturbance model based on the first initial sensor data comprises updating machine learning algorithms of the disturbance model. In some examples, updating the disturbance model based on the first initial sensor data comprises updating rule-based model of the disturbance model. In some examples, the first initial sensor data can be seen as subject specific sensor data. In some examples, the updating the disturbance model based on the first initial sensor data results in a subject specific disturbance model. The subject specific disturbance model for example comprises machine learning algorithms updated based on the second initial sensor data. The subject specific disturbance model for example comprises rule-based models updated based on the second initial sensor data. Advantageously, updating the disturbance model based on the first initial sensor data may result in improved accuracy of determination of disturbance data, and thus, an increased accuracy in instructions provided to the subject during and/or after the first cycle and/or second cycle. In some examples the electronic device is configured to generate artificial data, e.g., artificial sensor data, based upon which the disturbance model can be trained and/or updated. In some examples, the electronic device is configured to generate artificial data using GAD.

In one or more example electronic devices, the electronic device is configured to perform a second initial cycle optionally performed after the first initial cycle. In some examples, the second initial cycle is performed prior to the first cycle and/or second cycle. In some examples, the second initial cycle can be seen as a calibration cycle. In one or more example electronic devices, to perform the second initial cycle comprises to provide a second initial instruction indicative of a second action via the interface. The second initial instruction is for example provided (such as to the subject) via the interface. In some examples, the second action is an action carried out by the subject. The second initial instruction for example comprises an image, a video message, a text message and/or an audio message. The second initial instruction is for example provided via a display of an electronic device. The second initial instruction is for example provided via a speakerphone of an electronic device. The second initial instruction may be provided via a display and a speakerphone of an electronic device. In some examples, the second initial instruction may instruct the subject to create one or more artifacts in the second initial sensor data, e.g., by blinking eyes. For example, the second initial instruction may comprise the message “Please blink once every second for the next 10 seconds”. In some examples, the electronic device is configured to request user input indicative of the start of the second initial cycle. For example, the electronic device may be configured to start the second initial cycle in accordance with a user input requesting the start of the second initial cycle being received, e.g., the user pushes a “start first initial cycle” button provided by the interface of the electronic device”. In one or more example electronic devices, to perform the second initial cycle comprises to obtain second initial sensor data indicative of electrical activity of the brain of the subject. In some examples, the second action can be seen as an action associated with the second initial cycle. The second action for example comprises eye blinking, lateral eye movement, chewing, eye closing, jaw clenching, closed eyes for a time period, open eyes for a time period, talking and/or head movement performed by the subject. In one or more example electronic devices, to perform the second initial cycle comprises to determine whether the second initial sensor data is indicative of the second action. In some examples, the second initial sensor data is obtained via electrical sensors, e.g., electrodes, associated with an EEG cap. In some examples, the second sensor data can be seen as information indicative of the electrical activity of the brain of the subject. In one or more example electronic devices, to perform the second initial cycle comprises to, e.g. in accordance with a determination that the second initial sensor data is indicative of the second action, update the disturbance model based on the second initial sensor data. In some examples, updating the disturbance model based on the second initial sensor data comprises updating machine learning algorithms of the disturbance model. In some examples, updating the disturbance model based on the second initial sensor data comprises updating a rule-based model of the disturbance model. In some examples, the second initial sensor data can be seen as subject specific sensor data, e.g., the obtained second initial sensor data associated with the first action may change depending on the subject. In some examples, the electronic device is configured to update the disturbance model based on the second initial data such that the disturbance model is a subject specific disturbance model. The subject specific disturbance model for example comprises machine learning algorithms updated based on the second initial sensor data. The subject specific disturbance model for example comprises rule-based models updated based on the second initial sensor data. Advantageously, updating the disturbance model based on the first initial sensor data may result in improved accuracy of determination of disturbance data, and thus, an increased accuracy in instructions provided to the subject during and/or after the first cycle and/or second cycle. In some examples, each initial cycle is associated with a different action and/or related artifact performed by the subject. In other words, the electronic device may be configured to update, e.g., such as calibrate, the disturbance model based on different artifacts during each initial cycle.

In one or more example electronic devices, the first action is one or more of: eye blink, lateral eye movement, chew, close eyes, and jaw clench. In some examples, eye blink can be seen as the subject blinking. In some examples, lateral eye movement can be seen as the subject moving its eyes laterally (such as to the left or right). In some examples, chew can be seen as the subject moving its jaw (such as using muscles of mastication). In some examples, close eyes can be seen as the subject closing its eyes for a period of time (such as a period of time greater than blinking). In some examples, jaw clench can be seen as tensing the muscles of mastication.

In one or more example electronic devices, to determine disturbance data comprises to determine a probability vector indicative of the probability of a plurality of artifacts. In one or more example electronic devices, the first disturbance parameter is based on the probability vector. The probability vector for example comprises values indicative of the probability of a plurality of artifacts in the first sensor data. In some examples, the disturbance model comprises the probability vector. For example, the electronic device is configured to, using the probability vector, determine the disturbance data indicative of a plurality of artifacts in the sensor data. For example, the probability vector comprises a matrix of values indicative of the probability of a plurality of artifacts in the first sensor data, e.g., the probability that a peak in the sensor data is indicative of an epileptiform spike. In some examples, the probability vector is indicative of the probability of the cause, e.g., eye blinking, lateral eye movement, chewing, eye closing, and/or jaw clenching, of a plurality of artifacts. In some examples, the electronic device is configured to calculate the probability vector during the first initial cycle, second initial cycle, first cycle and/or second cycle.

In one or more example electronic devices, the electronic device is configured to determine whether the first disturbance data satisfies a first stop criterion. In one or more example electronic devices, the electronic device is configured to, in accordance with first disturbance data satisfying the first stop criterion, provide a first closing instruction indicative of the end of the first cycle. The first stop criterion can for example be seen as a quality criterion. In other words, if the disturbance data is indicative of a high frequency and/or magnitude of artifacts, e.g., first disturbance data indicative of low quality sensor data, the first stop criterion is, in some examples, satisfied. In some examples, the first stop criterion is a value associated with the time elapsed since the start of the first cycle. For example, electronic device may be configured such that the first stop criterion is satisfied 20 or 30 minutes after the start of the first cycle or when sensor data having sufficient time duration, such as 20 minutes, are obtained. In some examples, the first closing instruction is provided (such as to the subject) via the interface of the electronic device (such as the electronic device 10 of FIG. 1A). The first closing instruction for example comprises an image, a video message, a text message and/or an audio message. The first closing instruction is for example provided via a display of an electronic device. The first closing instruction is for example provided via a speakerphone of an electronic device. The first closing instruction may comprise a message indicative of the successful completion of the test. In other words, the first closing instruction may comprise a message indicative of no further cycles required and/or a message indicative of the end of the monitoring of electrical activity of a brain. For example, the first closing instruction may comprise the message, “Test complete, please remove the cap”. In some examples, the first closing instruction comprises a message indicative of the unsuccessful completion of the first cycle. In some examples, the first closing instruction comprises a message indicative of ending the first cycle and/or beginning a new cycle (such as the second cycle). In some examples, the first closing instruction comprises a request for the subject to modify actions, such as to reduce actions, e.g., to reduce the frequency and/or magnitude of artifacts. For example, the first closing instruction comprises the message, “Test unsuccessful due to unclear data. Please reduce muscle activity and continue”.

In one or more example electronic devices, the electronic device is configured to determine whether the second disturbance data satisfies a second stop criterion. In one or more example electronic devices, the electronic device is configured to, in accordance with second disturbance data satisfying the second stop criterion, provide a second closing instruction indicative of the end of the second cycle. The second stop criterion can for example be seen as a quality criterion. In other words, if the disturbance data is indicative of a high frequency and/or magnitude of artifacts, e.g., first disturbance data indicative of low quality sensor data, the second stop criterion is, in some examples, satisfied. In some examples, the second stop criterion is a value associated with the time elapsed since the start of the second cycle. For example, the electronic device may be configured such that the second stop criterion is satisfied 20 minutes after the start of the second cycle. In some examples, the second closing instruction is provided (such as to the subject) via the interface of the electronic device (such as the electronic device 10 of FIG. 1A). The second closing instruction for example comprises an image, a video message, a text message and/or an audio message. The second closing instruction is for example provided via a display of an electronic device and/or via a speakerphone of an electronic device. The second closing instruction may comprise a message indicative of the successful completion of the test. In other words, the second closing instruction may comprise a message indicative of no further cycles required and/or a message indicative of the end of the monitoring of electrical activity of a brain. For example, the second closing instruction may comprise the message, “Test complete, please remove the cap”. In some examples, the second closing instruction comprises a message indicative of the unsuccessful completion of the first cycle. In some examples, the second closing instruction comprises a message indicative of ending the second cycle and/or beginning a new cycle. In some examples, the second closing instruction comprises a request for the subject to modify actions, such as to reduce actions, e.g., to reduce the frequency and/or magnitude of artifacts. For example, the second closing instruction comprises the message, “Test unsuccessful due to unclear data. Please reduce muscle activity and continue”.

In one or more example electronic devices, to determine, using a disturbance model, first disturbance data including a first disturbance parameter based on the first sensor data comprises processing the first disturbance data using a 30 second sliding window with 1 second step size. In some examples, the sliding window comprises sensor data obtained by the EEG cap. For example, the sliding window can be seen as a time period. For example, the start of the 30 second sliding window may be associated with the present moment. For example, the end of the 30 second sliding window may be associated with the moment 30 seconds before the present moment. In some examples, the sliding window moves forwards in time by 1 second every second.

In one or more example electronic devices, the electronic device is configured to sample sensor data at a frequency greater than 100 Hz. For example, the electronic device is configured to sample sensor data at a frequency between 100 Hz and 500 Hz. For example, the electronic device is configured to sample sensor data at a frequency between 200 Hz and 300 Hz. For example, the electronic device is configured to sample sensor data at a frequency of 256 Hz.

In one or more example electronic devices, the first disturbance data is based on one or more first primary disturbance parameters associated with a primary artifact and one or more first secondary disturbance parameters associated with a secondary artifact. In some examples, the primary disturbance parameter is associated with the first primary disturbance criterion and/or the second primary disturbance criterion. The primary disturbance parameter is for example associated with a frequency of the primary artifact. The primary disturbance parameter is for example associated with a magnitude, e.g., intensity of the primary artifact. In some examples, the secondary disturbance parameter is associated with the first secondary disturbance criterion and/or the second secondary disturbance criterion. The secondary disturbance parameter is for example an artifact associated with a frequency of the secondary artifact. The secondary disturbance parameter is for example an artifact associated with a magnitude, e.g., intensity, of the secondary artifact. The primary artifact is for example a different artifact to the second artifact. In some examples, the primary artifact and secondary artifact are associated with the same cycle (such as the first cycle or the second cycle).

A method of monitoring electrical activity in a brain is disclosed. The method is performed by an electronic device. In one or more examples, the electronic device comprises an interface and/or one or more processors.

In one or more examples, the method comprises performing a first cycle. In one or more example methods, performing the first cycle may comprise obtaining first sensor data indicative of electrical activity of a brain of a subject. In one or more example methods, performing the first cycle may comprise determining, using a disturbance model, first disturbance data including a first disturbance parameter based on the first sensor data. In one or more example methods, determining first disturbance data comprises determining the first disturbance parameter over a first time window. In one or more example methods, the first time window is in the range from 10 seconds to 1 minute. Performing the first cycle may comprise determining whether the first disturbance data satisfies one or more first disturbance criteria. In one or more example methods, determining whether the first disturbance data satisfies one or more first disturbance criteria comprises determining whether the first disturbance data satisfies a first primary disturbance criterion of the one or more first disturbance criteria. In one or more example methods, performing the first cycle may comprise, in accordance with a determination that the first primary disturbance criterion of the one or more first disturbance criteria is satisfied, providing a first primary instruction via the interface. In one or more example methods, determining whether the first disturbance data satisfies one or more first disturbance criteria comprises determining whether the first disturbance data satisfies a first secondary disturbance criterion of the one or more first disturbance criteria. In one or more example methods, in accordance with a determination that the first secondary disturbance criterion of the one or more first disturbance criteria is satisfied, providing a first secondary instruction via the interface.

In one or more example methods, the method comprises, after performing the first cycle, performing a second cycle. In one or more example methods, performing the second cycle comprises obtaining second sensor data indicative of electrical activity of the brain of the subject. In one or more example methods, performing the second cycle comprises determining, using the disturbance model, second disturbance data including a second disturbance parameter based on the second sensor data. In one or more example methods, determining second disturbance data comprises determining a second disturbance parameter over a second time window. In one or more example methods, the second time window is in the range from 10 seconds to 1 minute. In one or more example methods, performing the second cycle comprises determining whether the second disturbance data satisfies one or more second disturbance criteria. In one or more example methods, determining whether the second disturbance data satisfies one or more second disturbance criteria comprises determining whether the second disturbance data satisfies a second primary disturbance criterion of the one or more second disturbance criteria. In one or more example methods, performing the second cycle comprises, in accordance with a determination that the second primary disturbance criterion of the one or more second disturbance criteria is satisfied, providing a second primary instruction via the interface. In one or more example methods, determining whether the second disturbance data satisfies one or more second disturbance criteria comprises determining whether the second disturbance data satisfies the second secondary disturbance criterion of the one or more second disturbance criteria. In one or more example methods, the method comprises, in accordance with a determination that the second secondary disturbance criterion of the one or more second disturbance criteria is satisfied, providing a second secondary instruction via the interface.

In one or more example methods, the method comprises performing a first initial cycle. In one or more example methods, performing the first initial cycle comprises providing a first initial instruction indicative of a first action via the interface. In one or more example methods, performing the first initial cycle comprises obtaining first initial sensor data indicative of electrical activity of the brain of the subject. In one or more example methods, performing the first initial cycle comprises determining whether the first initial sensor data is indicative of the first action. In one or more example methods, performing the first initial cycle comprises, in accordance with a determination that the first initial sensor data is indicative of the first action, updating the disturbance model based on the first initial sensor data.

In one or more example methods, the method comprises performing a second initial cycle. In one or more example methods, performing the second initial cycle comprises providing a second initial instruction indicative of a second action via the interface. In one or more example methods, performing the second initial cycle comprises obtaining second initial sensor data indicative of electrical activity of the brain of the subject. In one or more example methods, performing the second initial cycle comprises determining whether the second initial sensor data is indicative of the second action. In one or more example methods, performing the second initial cycle comprises, in accordance with a determination that the second initial sensor data is indicative of the second action, updating the disturbance model based on the second initial sensor data.

In one or more example methods, the first action is one or more of: eye blink, lateral eye movement, chew, close eyes, and jaw clench.

In one or more example methods, determining disturbance data comprises determining a probability vector indicative of the probability of a plurality of artifacts. In one or more example methods the first disturbance parameter is based on the probability vector.

In one or more example methods, the method comprises determining whether the first disturbance data satisfies a first stop criterion. In one or more example methods, the method comprises, in accordance with first disturbance data satisfying the first stop criterion, providing a first closing instruction indicative of the end of the first cycle.

In one or more example methods, the method comprises determining whether the second disturbance data satisfies a second stop criterion. In one or more example methods, the method comprises, in accordance with second disturbance data satisfying the second stop criterion, providing a second closing instruction indicative of the end of the second cycle.

In one or more example methods, determining, using a disturbance model, first disturbance data including a first disturbance parameter based on the first sensor data comprises processing the first disturbance data using a 30 second sliding window with 1 second step size.

In one or more example methods, the method comprises sampling sensor data at a frequency greater than 100 Hz.

In one or more example methods, the first disturbance data is based on one or more first primary disturbance parameters associated with a primary artifact and one or more first secondary disturbance parameters associated with a secondary artifact.

The figures are schematic and simplified for clarity, and they merely show details which aid understanding the disclosure, while other details have been left out. Throughout, the same reference numerals are used for identical or corresponding parts.

It is to be understood that a description of a feature in relation to the data collector, the cap, and/or the electronic device is also applicable to the corresponding feature in the method(s) of operating an electronic device as disclosed herein.

FIG. 1A is a diagram illustrating an example subject 12, electrodes 13 of the EEG cap, EEG cap 14, data collector 16, electronic device 18, server 20, and electronic device 10. The electronic device 10 for example comprises the data collector 16 and/or the electronic device 18. The electronic device 18 is for example a smartphone or a tablet computer.

The EEG for example comprises between 15 and 45 electrodes 13. The EEG for example comprises between 20 and 30 electrodes 13. The EEG cap 14 for example comprises 27 electrodes 13. In some examples, data is provided 15 from the EEG cap 14 to the data collector 16 via a wired connection. In some examples, data is provided from the EEG cap 14 to the data collector 16 via a wireless connection (e.g., via Bluetooth and/or Near Field Communication (NFC)). In some examples, the data collector 16 can be seen as an amplifier.

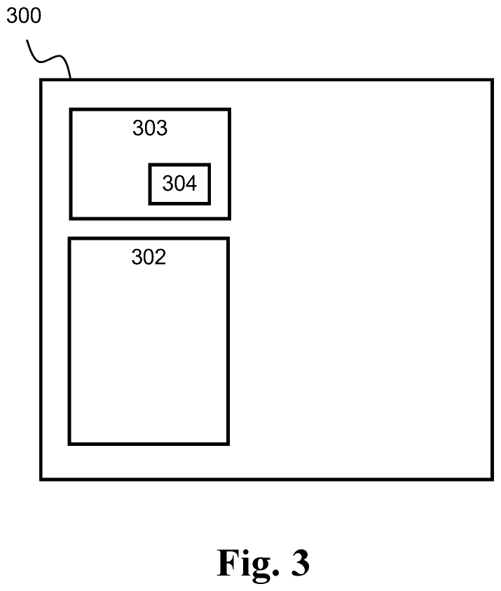

In some examples, the data collector 16 provides sensor data to the electronic device 18 via communication link 17. In some examples, the data collector 16 receives data from the electronic device 18 via communication link 17. In some examples, the electronic device 18 provides data to the data collector 16 via communication link 17. In some examples, the electronic device 18 receives data from the data collector 16 via communication link 17. In some examples, the communication link 17 is a wired connection. In some examples, the communication link 17 is a wireless connection (e.g., Bluetooth and/or NFC). The data collector 16 and/or the electronic device 18 may comprise processor 302 of FIG. 3. In some examples, the electronic device 18 provides data to a server 20 via the communication link 19. In some examples, the electronic device 18 receives data from a server 20 via the communication link 19. In some examples, the server 20 provides data to an electronic device 18 via the communication link 19. In some examples, the server 20 provides data to an electronic device 18 via the communication link 19. In some examples, the communication link 19 is wired. In some examples, the communication link 19 is a wireless connection, e.g., Bluetooth and/or NFC. In some examples, the communication link 19 uses Hypertext Transfer Protocol Secure (HTTPS). In some examples, the data transfer across the communication link occurs across a cloud network. For example, the server 20 can store the data initially provided by the EEG cap 14. In one or more examples, the data uploaded to the server 20 is accessible via an electronic device different to electronic device 18. For example, the data can be accessed by a medical professional via a device connected to the cloud storage network 20.

FIG. 1B shows a flow diagram of an exemplary method, performed by an electronic device comprising an interface and one or more processors, of monitoring electrical brain activity according to the disclosure.

The method 100 comprises performing 150 N initial cycles including a first initial cycle where i is an index indicating the i′th initial cycle. N may be in the range from 1 to 10, such as in the range from 3 to 7. For the first initial cycle, i is initialised by setting i=1. Thus, the first initial cycle can be seen as the cycle where i=1. For example, the second initial cycle can be seen as i=2.

In method 100, performing an i′th initial cycle comprises initializing and/or incrementing S101 the index i. For the first initial cycle, the index i is initialised and for the following initial cycles, the index i is incremented in S101.

In method 100, performing N initial cycles comprises providing S102 an i′th initial instruction (II_i) indicative of an i′th action via the interface, e.g. by displaying the i′th initial instruction on a display and/or outputting audio representative of the i′th initial instruction, and obtaining S104 i′th initial sensor data (ISD_i) indicative of electrical activity of the brain of the subject, e.g. in accordance with detecting an input indicating that the user performs the i′th action.

In the method 100, performing N initial cycles optionally comprises determining S106 whether the i′th initial sensor data is indicative of the i′th action, and in accordance with a determination that the i′th initial sensor data is indicative of the i′th initial action, updating S108 the disturbance model (DM) based on the i′th initial sensor data. In other words, the method 100 optionally does not proceed to an update of the disturbance model using the i′th initial sensor data if the i′th initial sensor data recorded does not reflect the required action, which in turn leads to improved and more accurate disturbance model. In one or more examples the validity check in S106 and conditional update of the DM may be omitted, i.e. the method may proceed to S108 directly from S104.

In accordance with a determination that the i′th initial sensor data is not indicative of the i′th action, the method 100 proceeds to, forgoing updating the disturbance model based on the i′th initial sensor data and/or providing S107 feedback to the user indicative of the i′th initial action not being completed correctly. From S107, the method optionally returns to S101 as shown, where index i may be incremented to proceed to next initial cycle, or to S102 (not shown) for repeating the i′th initial cycle. In one or more example methods, an i′th initial cycle may be repeated only once before proceeding to the next initial cycle by incrementing index i.

In method 100, performing N initial cycles comprises determining S106A whether a required number of initial cycles have been performed e.g. by determining whether index i=N. Other criteria for determining if the required number of initial cycles have been performed may be applied. For example, one or more quality parameters indicative of quality of respective initial sensor data ISD_i may be determined and used to determine whether a required number of initial cycles have been performed. For example, one or more time parameters may be determined and used to determine whether a required number of initial cycles have been performed, e.g. if a subject has worn the EEG too long or the initial cycles have taken too long time. In one or more example methods, in accordance with determining S106A that the required number of cycles have not been performed, the method returns to initializing and/or incrementing S101 the index i to proceed to the next initial cycle.

In one or more example methods, updating S108 the disturbance model (DM) may be performed after S106A. In this case, the disturbance model may be updated based on the valid initial sensor data of ISD_i for 1, . . . , N, i.e. the initial sensor data that were determined to reflect the respective initial actions. In other words, the method may comprise updating S108 the disturbance model after all initial cycles have been completed.

When the required number of initial cycles have been performed, the method 100 proceeds to performing a first cycle, wherein performing the first cycle comprises obtaining S118 first sensor data (SD_1) indicative of electrical activity of a brain of a subject and determining S120, using the disturbance model DM, first disturbance data DD_1 including a first disturbance parameter based on the first sensor data. Performing the first cycle comprises determining S126 whether the first disturbance data satisfies one or more first disturbance criteria, and in accordance with a determination that a first disturbance criterion of the one or more first disturbance criteria is satisfied, omit or mark the disturbed first sensor data from the first sensor data and/or providing S127 a first instruction via the interface, such as providing S128 a first primary instruction in accordance with a determination that a first primary disturbance criterion of the one or more first disturbance criteria is satisfied and/or providing S130 a first secondary instruction in accordance with a determination that a first secondary disturbance criterion of the one or more first disturbance criteria is satisfied. Performing the first cycle may comprise, after providing S127 a first instruction, return to obtaining S118 first sensor data (SD_1) indicative of electrical activity of a brain of a subject. Performing the first cycle may comprise, in accordance with a determination that none of the first disturbance criteria are satisfied, determining S129 whether the first cycle is complete. Performing the first cycle may comprise, in accordance with a determination that the first cycle is not complete, return to obtaining S118 first sensor data (SD_1) indicative of electrical activity of a brain of a subject. Performing the first cycle may comprise, in accordance with a determination that the first cycle is complete, outputting S131, such as storing in memory and/or transmitting e.g. to server device, the first sensor data SD_1.

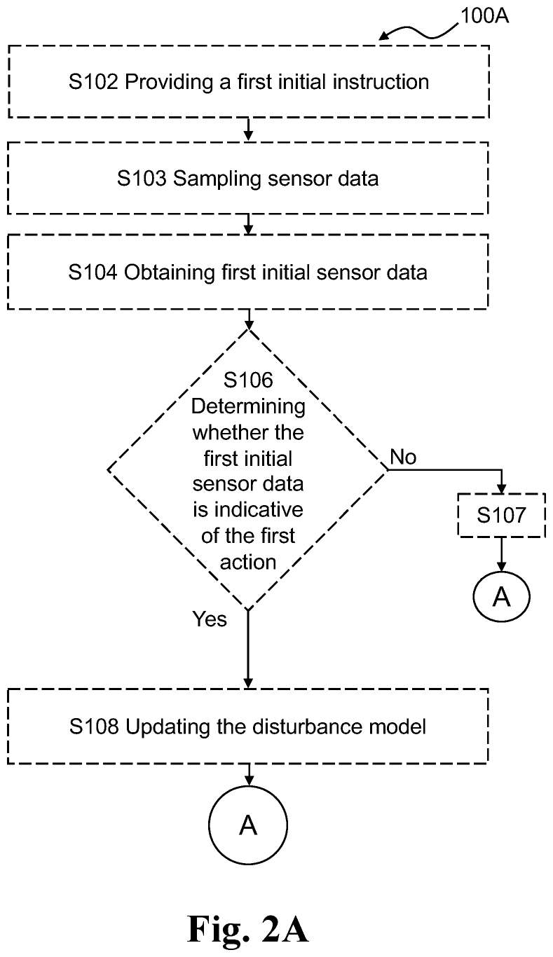

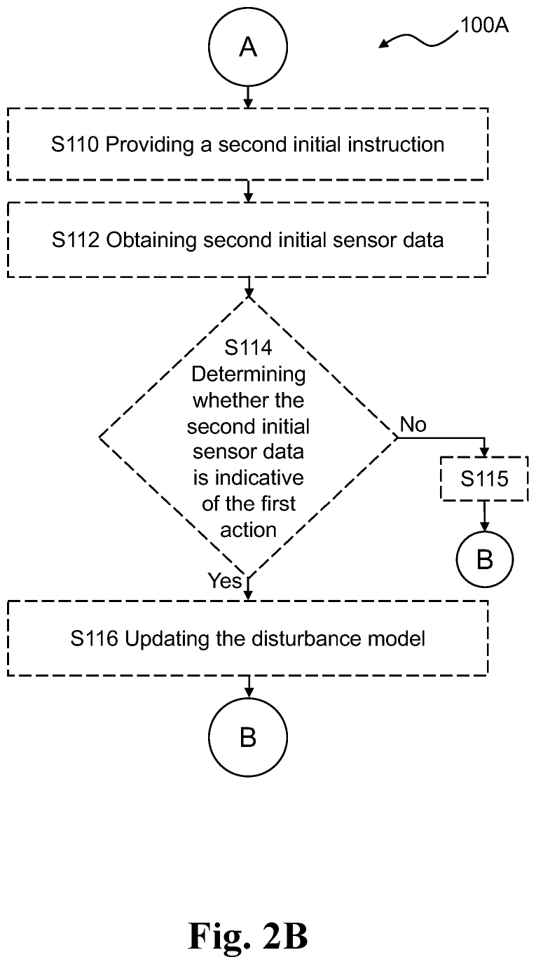

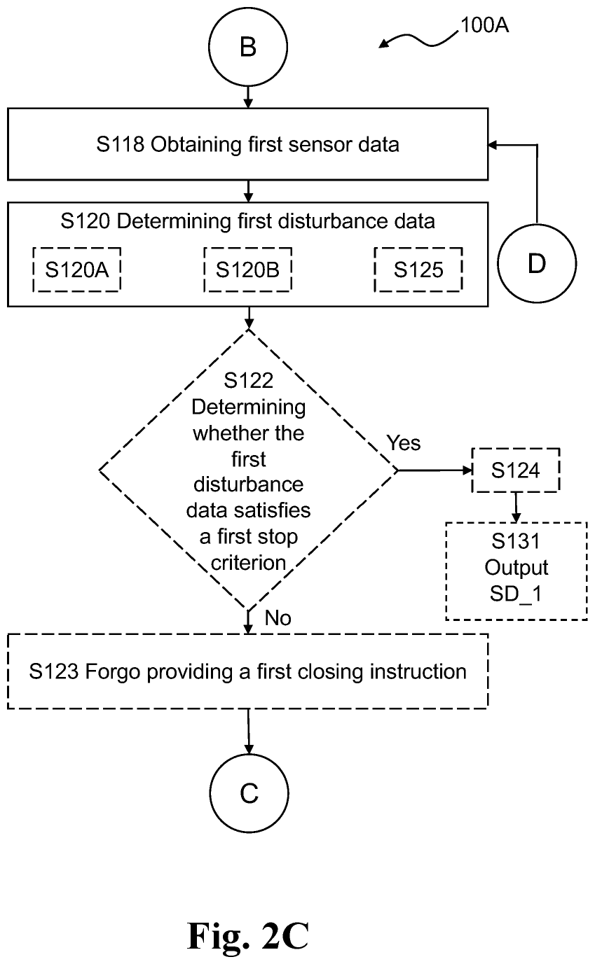

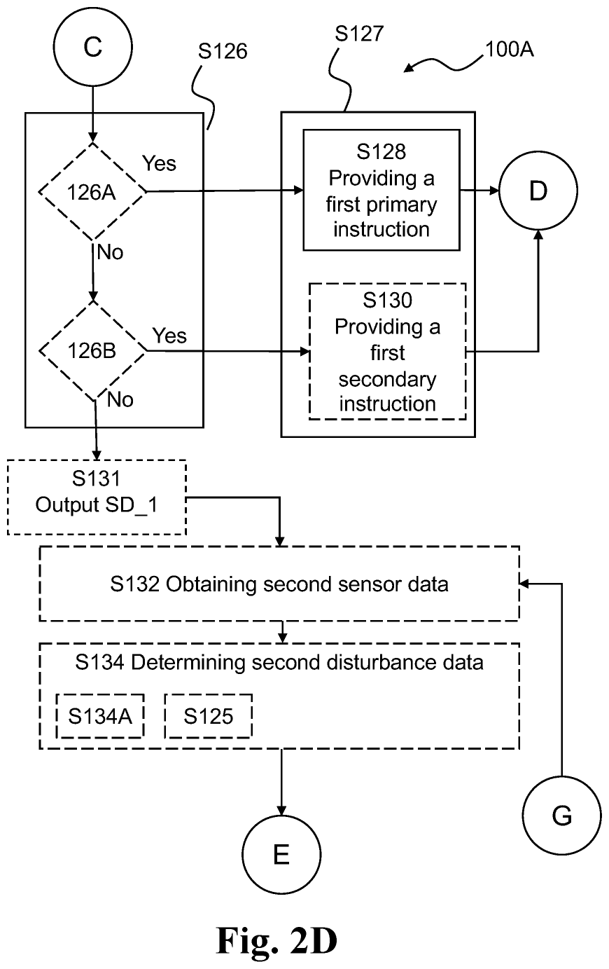

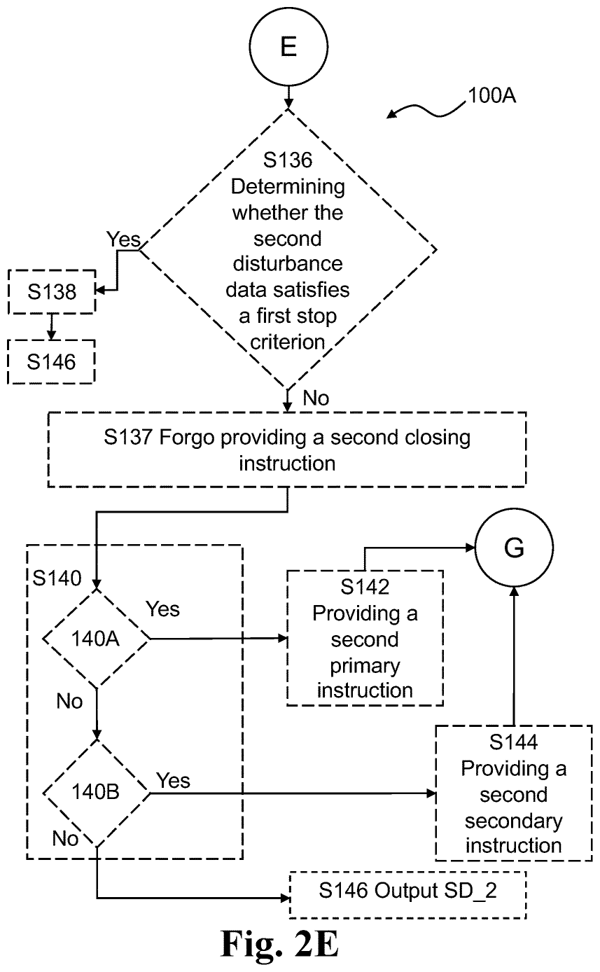

FIGS. 2A-E show a flow diagram of an exemplary method, performed by an electronic device comprising an interface and one or more processors, of monitoring electrical activity of a brain according to the disclosure.

In one or more examples, the method 100A comprises performing a first cycle. Performing the first cycle may comprise obtaining S118 first sensor data indicative of electrical activity of a brain of a subject. Performing the first cycle may comprise determining S120, using a disturbance model, first disturbance data including a first disturbance parameter based on the first sensor data. In one or more example methods, determining S120 first disturbance data comprises determining S120A the first disturbance parameter over a first time window. In one or more example methods, the first time window is in the range from 10 seconds to 1 minute. For example, the first disturbance parameter may comprise a number of first artifacts determined within the first time window, e.g., 30 seconds.

Performing the first cycle may comprise determining S126 whether the first disturbance data satisfies one or more first disturbance criteria. In one or more example methods, determining S126 whether the first disturbance data satisfies one or more first disturbance criteria comprises determining S126A whether the first disturbance data satisfies a first primary disturbance criterion of the one or more first disturbance criteria. Performing the first cycle may comprise, in accordance with a determination that the first primary disturbance criterion of the one or more first disturbance criteria is satisfied, providing S128 a first primary instruction via the interface. In one or more example methods, in accordance determining with a determination that the first primary disturbance criterion of the one or more first disturbance criteria is not satisfied, forgoing providing a first primary instruction via the interface. In one or more example methods, determining S126 whether the first disturbance data satisfies one or more first disturbance criteria comprises determining S126B whether the first disturbance data satisfies a first secondary disturbance criterion of the one or more first disturbance criteria. In one or more example methods, in accordance with a determination that the first secondary disturbance criterion of the one or more first disturbance criteria is satisfied, providing S130 a first secondary instruction via the interface. In one or more example methods, the method comprises, in accordance with a determination that the first secondary disturbance criterion of the one or more first disturbance criteria is not satisfied, forgoing providing a first secondary instruction via the interface. In some examples, the method comprises repeating the first cycle one or more times. In some examples, the method comprises forgoing repeating the first cycle.

In one or more example methods, the method comprises, after performing the first cycle, performing a second cycle. In one or more example methods, performing the second cycle comprises obtaining S132 second sensor data indicative of electrical activity of the brain of the subject. In one or more example methods, performing the second cycle comprises determining S134, using the disturbance model, second disturbance data including a second disturbance parameter based on the second sensor data. In one or more example methods, determining S134 second disturbance data comprises determining S134A a second disturbance parameter over a second time window. In one or more example methods, the second time window is in the range from 10 seconds to 1 minute. In one or more example methods, performing the second cycle comprises determining S140 whether the second disturbance data satisfies one or more second disturbance criteria. In one or more example methods, determining S140 whether the second disturbance data satisfies one or more second disturbance criteria comprises determining S140A whether the second disturbance data satisfies a second primary disturbance criterion of the one or more second disturbance criteria. In one or more example methods, performing the second cycle comprises, in accordance with a determination that the second primary disturbance criterion of the one or more second disturbance criteria is satisfied, providing S142 a second primary instruction via the interface. In one or more example methods, performing the second cycle comprises, in accordance with a determination that the second primary disturbance criterion of the one or more second disturbance criteria is not satisfied, forgoing providing a second primary instruction via the interface. In one or more example methods, determining S140 whether the second disturbance data satisfies one or more second disturbance criteria comprises determining S140B whether the second disturbance data satisfies the second secondary disturbance criterion of the one or more second disturbance criteria. In one or more example methods, the method comprises, in accordance with a determination that the second secondary disturbance criterion of the one or more second disturbance criteria is satisfied, providing S144 a second secondary instruction via the interface. In one or more example methods, performing the second cycle comprises, in accordance with a determination that the second secondary disturbance criterion of the one or more second disturbance criteria is not satisfied, forgoing providing a second secondary instruction via the interface.

In one or more example methods, the method comprises performing a first initial cycle. In one or more example methods, performing the first initial cycle comprises providing S102 a first initial instruction indicative of a first action via the interface. In one or more example methods, performing the first initial cycle comprises obtaining S104 first initial sensor data indicative of electrical activity of the brain of the subject. In one or more example methods, performing the first initial cycle comprises determining S106 whether the first initial sensor data is indicative of the first action. In one or more example methods, performing the first initial cycle comprises, in accordance with a determination that the first initial sensor data is indicative of the first action, updating S108 the disturbance model based on the first initial sensor data. In one or more example methods, performing the first initial cycle comprises, in accordance with a determination that the first initial sensor data is not indicative of the first action, forgoing updating the disturbance model based on the first initial sensor data. In one or more example methods, performing the first initial cycle comprises, in accordance with a determination that the first initial sensor data is not indicative of the first action, providing S107 feedback indicative of the first initial action not being completed correctly. In one or more example methods, performing the first initial cycle comprises, in accordance with a determination that the first initial sensor data is not indicative of the first action, providing S102 a first initial instruction indicative of a first action via the interface.

In one or more example methods, the method comprises performing a second initial cycle. In one or more example methods, performing the second initial cycle comprises providing S110 a second initial instruction indicative of a second action via the interface. In one or more example methods, performing the second initial cycle comprises obtaining S112 second initial sensor data indicative of electrical activity of the brain of the subject. In one or more example methods, performing the second initial cycle comprises determining S114 whether the second initial sensor data is indicative of the second action. In one or more example methods, performing the second initial cycle comprises, in accordance with a determination that the second initial sensor data is indicative of the second action, updating S116 the disturbance model based on the second initial sensor data. In one or more example methods, performing the second initial cycle comprises, in accordance with a determination that the second initial sensor data is not indicative of the second action, forgoing updating the disturbance model based on the second initial sensor data. In one or more example methods, performing the second initial cycle comprises, in accordance with a determination that the second initial sensor data is not indicative of the second action, providing S115 feedback indicative of the second initial action not being completed correctly. In one or more example methods, performing the second initial cycle comprises, in accordance with a determination that the second initial sensor data is not indicative of the second action, providing S102 a second initial instruction indicative of a second action via the interface.

In one or more example methods, the first action is one or more of: eye blink, lateral eye movement, chew, close eyes, and jaw clench.

In one or more example methods, determining S120 first disturbance data and/or determining S134 second disturbance data comprises determining S125 a probability vector indicative of the probability of a plurality of artifacts. In one or more example methods the first disturbance parameter is based on the probability vector.

In one or more example methods, the method comprises determining S122 whether the first disturbance data satisfies a first stop criterion. In one or more example methods, the method comprises, in accordance with first disturbance data satisfying the first stop criterion, providing S124 a first closing instruction indicative of the end of the first cycle. In one or more example methods, the method comprises, in accordance with first disturbance data not satisfying the first stop criterion, forgoing S123 providing a first closing instruction indicative of the end of the first cycle.

In one or more example methods, the method comprises determining S136 whether the second disturbance data satisfies a second stop criterion. In one or more example methods, the method comprises, in accordance with second disturbance data satisfying the second stop criterion, providing S138 a second closing instruction indicative of the end of the second cycle. In one or more example methods, the method comprises, in accordance with second disturbance data not satisfying the second stop criterion, forgoing providing S137 a second closing instruction indicative of the end of the second cycle.

In one or more example methods, determining S120, using a disturbance model, first disturbance data including a first disturbance parameter based on the first sensor data comprises processing S120B the first disturbance data using a 30 second sliding window with 1 second step size.

In one or more example methods, the method comprises sampling S103 sensor data at a frequency greater than 100 Hz.

In one or more example methods, the first disturbance data is based on one or more first primary disturbance parameters associated with a primary artifact and one or more first secondary disturbance parameters associated with a secondary artifact.

Performing the second cycle may comprise, e.g. in accordance with a determination that the second disturbance data does not satisfy any second disturbance criterion and/or after providing S138 the second closing instruction, outputting S146, such as storing in memory and/or transmitting e.g. to server device, the second sensor data SD_2. In one or more examples, where the method comprises performing the second cycle, outputting S131 the first sensor data may take place after S140.

FIG. 3 shows a block diagram of an exemplary electronic device 300 according to the disclosure. The electronic device 300 comprises processor circuitry 302 and an interface 303. In some examples, the interface 303 comprises a display 304, e.g., a display screen or a touch-screen. The electronic device 300 is configured to perform any of the methods disclosed in FIG. 2. In other words, the electronic device 300 is configured for monitoring electrical activity of a brain. The electronic device 300 may be embodied as electronic device 18.

An electronic device 300 is disclosed. The electronic device 300 comprises an interface and one or more processors. The electronic device 300 is configured to perform, e.g., using processor circuitry 302, a first cycle. The first cycle may be referred to as a first EEG capture cycle. In one or more example electronic devices, to perform the first cycle comprises to obtain, e.g., using processor circuitry 302 and/or interface 303, first sensor data indicative of electrical activity of a brain of a subject. In one or more example electronic devices, to perform the first cycle comprises to determine, e.g., using processor circuitry 302, using a disturbance model, first disturbance data including a first disturbance parameter based on the first sensor data. In some examples, the first disturbance data can be seen as information indicative of an artifact, e.g., a disturbance. An artifact can for example be seen in the first sensor data as a spike, peak etc. An artifact may be seen as noise in the first sensor data. For example, the first disturbance parameter may comprise information indicative of the number of eye blinks, lateral eye movements, chews, eye closings, and/or jaw clenches based on the first sensor data. In one or more example electronic devices, to perform the first cycle comprises to determine, e.g., using processor circuitry 302, whether the first disturbance data satisfies one or more first disturbance criteria. In one or more example electronic devices, to perform the first cycle comprises to, in accordance with a determination that a first primary disturbance criterion of the one or more first disturbance criteria is satisfied, provide, e.g., using processor circuitry 302, interface 303 and/or display 304, a first primary instruction via the interface.

The electronic device may be configured to, in accordance with a determination that a first secondary disturbance criterion of the one or more first disturbance criteria is satisfied, provide, e.g., using processor circuitry 302, interface 303 and/or display 304, a first secondary instruction via the interface. The first secondary disturbance criterion can be associated with the number of blinks, number of lateral eye movements, number of chews, number of times eyes closed, and/or number of times jaw clenched per unit time. For example, the first secondary instruction may comprise a request for the subject to modify actions, such as to reduce actions (e.g., to reduce the frequency of artifacts). For example, the first secondary instruction comprises a message instructing the subject to reduce frequency of actions, e.g., to blink less.

The electronic device can be configured to, after performing the first cycle, perform, e.g., using processor circuitry 302, a second cycle. To perform the second cycle comprises to obtain, e.g., using processor circuitry 302 and/or interface 303, second sensor data indicative of electrical activity of the brain of the subject. To perform the second cycle may comprise to determine, e.g., using processor circuitry 302, using the disturbance model, second disturbance data including a second disturbance parameter based on the second sensor data. In some examples, the second disturbance data can be seen as information indicative of an artifact, e.g., a disturbance. In some examples, an artifact can be seen as noise in the second sensor data. In some examples, an action performed by the subject can produce an artifact in the second sensor data. In some examples, the second disturbance parameter may comprise information indicative of the number of eye blinks, lateral eye movements, chews, eye closings, and/or jaw clenches based on the second sensor data. To perform the second cycle may comprise to determine, e.g., using processor circuitry 302, whether the second disturbance data satisfies one or more second disturbance criteria. To perform the second cycle may comprise to in accordance with a determination that a second primary disturbance criterion of the one or more second disturbance criteria is satisfied, provide, e.g., using processor circuitry 302, interface 303 and/or display 304, a second primary instruction via the interface.

The electronic device can be configured to, in accordance with a determination that a second secondary disturbance criterion of the one or more second disturbance criteria is satisfied, provide, e.g., using processor circuitry 302, interface 303 and/or display 304, a second secondary instruction via the interface.

The electronic device may be configured to perform, e.g., using processor circuitry 302, a first initial cycle. To perform the first initial cycle may comprise to provide, e.g., using processor circuitry 302, interface 303 and/or display 304, a first initial instruction indicative of a first action via the interface. To perform the first initial cycle may comprise to obtain, e.g., using processor circuitry 302 and/or interface 303, first initial sensor data indicative of electrical activity of the brain of the subject. To perform the first initial cycle may comprise to determine, e.g., using processor circuitry 302, whether the first initial sensor data is indicative of the first action. To perform the first initial cycle may comprise to in accordance with a determination that the first initial sensor data is indicative of the first action, update, e.g., using processor circuitry 302, the disturbance model based on the first initial sensor data.

The electronic device can be configured to perform, e.g., using processor circuitry 302, a second initial cycle. To perform the second initial cycle comprises to provide, e.g., using processor circuitry 302, interface 303 and/or display 304, a second initial instruction indicative of a second action via the interface. To perform the second initial cycle may comprise to obtain, e.g., using processor circuitry 302 and/or interface 303, second initial sensor data indicative of electrical activity of the brain of the subject. To perform the second initial cycle may comprise to determine, e.g., using processor circuitry 302, whether the second initial sensor data is indicative of the second action. To perform the second initial cycle may comprise to, in accordance with a determination that the second initial sensor data is indicative of the second action, update, e.g., using processor circuitry 302, the disturbance model based on the second initial sensor data.

The first action may be one or more of: eye blink, lateral eye movement, chew, close eyes, and jaw clench.

To determine first disturbance data may comprise to determine, e.g., using processor circuitry 302, the first disturbance parameter over a first time window. The first time window may be in the range from 10 seconds to 1 minute. The first time window is for example in the range from 20 seconds to 40 seconds. The first time window is for example in the range from 0.5 seconds to 30 seconds.

To determine second disturbance data may comprise to determine, e.g., using processor circuitry 302, a second disturbance parameter over a second time window. In one or more example devices, the second time window is in the range from 10 seconds to 1 minute. The second time window is for example in the range from 20 seconds to 40 seconds. The second time window is for example in the range from 0.5 seconds to 30 seconds.

To determine disturbance data may comprise to determine, e.g., using processor circuitry 302, a probability vector indicative of the probability of a plurality of artifacts. The first disturbance parameter can be based on the probability vector.

The electronic device may be configured to determine, e.g., using processor circuitry 302, whether the first disturbance data satisfies a first stop criterion; the electronic device can be configured to, in accordance with first disturbance data satisfying the first stop criterion, provide, e.g., using processor circuitry 302, interface 303 and/or display 304, a first closing instruction indicative of the end of the first cycle.

The electronic device may be configured to determine, e.g., using processor circuitry 302, whether the second disturbance data satisfies a second stop criterion. The electronic device may be configured to, in accordance with second disturbance data satisfying the second stop criterion, provide, e.g., using processor circuitry 302, interface 303 and/or display 304, a second closing instruction indicative of the end of the second cycle.

To determine, using a disturbance model, first disturbance data including a first disturbance parameter based on the first sensor data may comprise processing, e.g., using processor circuitry 302, the first disturbance data using a 30 second sliding window with 1 second step size.

The electronic device may be configured to sample, e.g., using processor circuitry 302, sensor data at a frequency greater than 100 Hz.

The first disturbance data can be based on one or more first primary disturbance parameters associated with a primary artifact and one or more first secondary disturbance parameters associated with a secondary artifact.

The processor circuitry 302 is optionally configured to perform any of the operations disclosed in FIG. 2 (such as any one or more of: S102, S103, S104, S106, S107, S108, S110, S112, S114, S115, S116, S118, S120, S120A, S120B, S122, S123, S124, S125, S126, S126A, S126B, S128, S130, S132, S134, S134A, S136, S137, S138, S140, S140A, S140B, S142, S144). The operations of the electronic device 300 may be embodied in the form of executable logic routines (e.g., lines of code, software programs, etc.) that are stored on a non-transitory computer readable medium (e.g., the memory circuitry 301) and are executed by the processor circuitry 302).

Furthermore, the operations of the electronic device 300 may be considered a method that the electronic device 300 is configured to carry out. Also, while the described functions and operations may be implemented in software, such functionality may as well be carried out via dedicated hardware or firmware, or some combination of hardware, firmware and/or software.

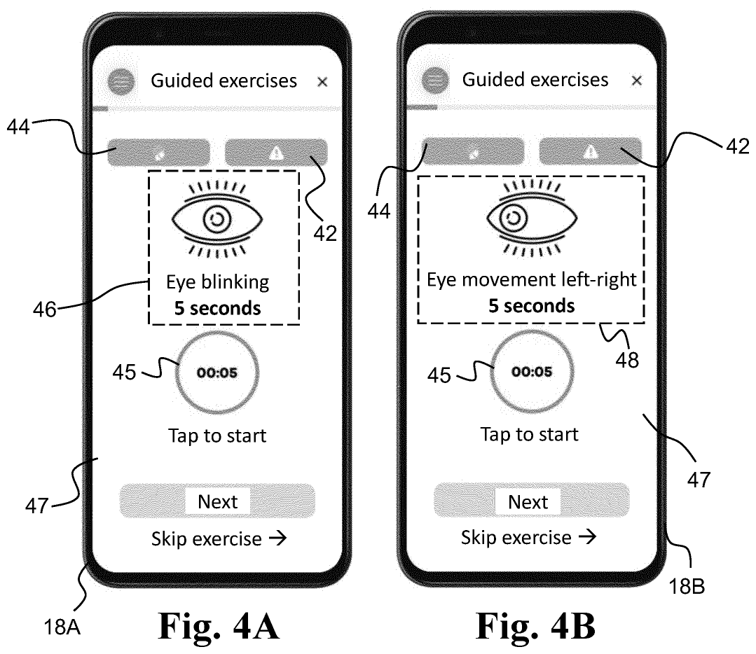

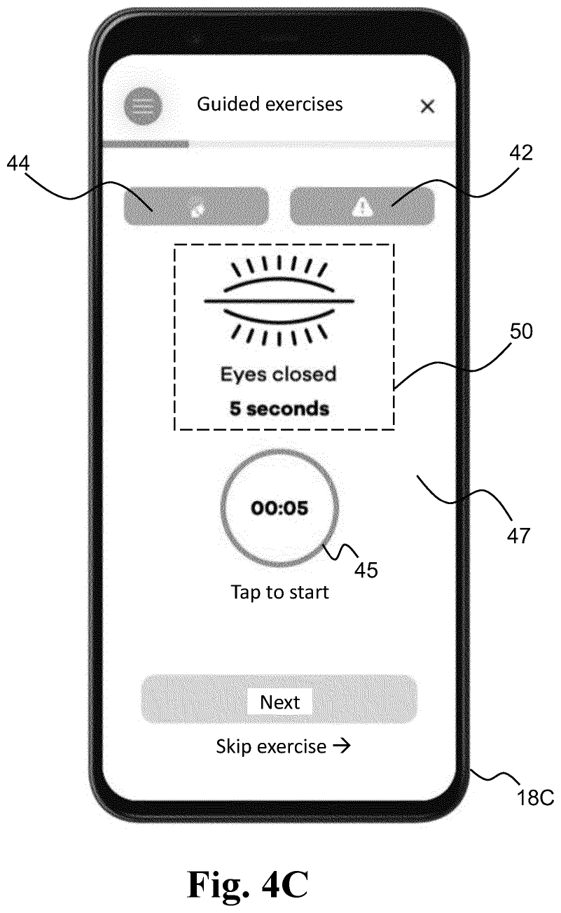

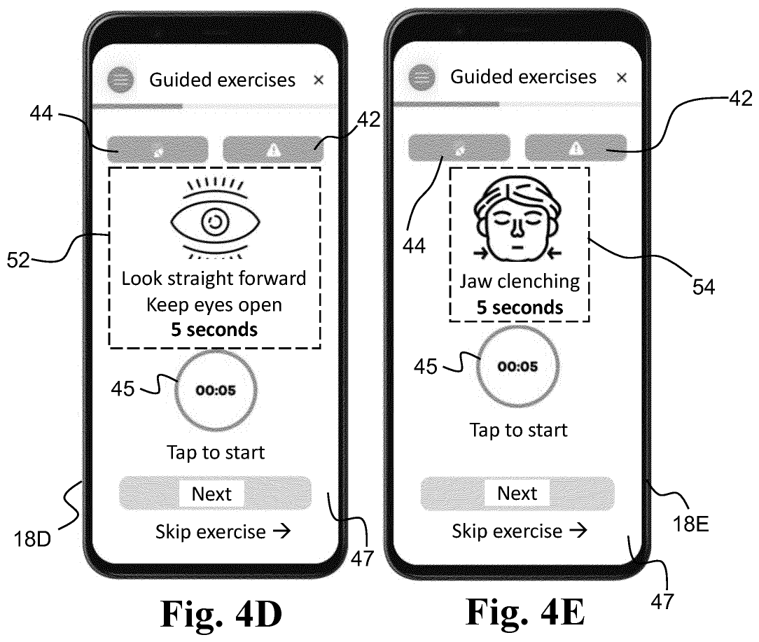

FIG. 4A shows example user interfaces of the electronic device 18 during EEG capture.

Electronic devices 18A, 18B, 18C, 18D, 18E may be referred to individually as electronic device 18A, electronic device 18B, electronic device 18C, electronic device 18D, electronic device 18E and collectively as electronic device 18. For example, electronic devices 18A, 18B, 18C, 18D and 18E can be seen as the same electronic device 18 during different initial cycles, e.g., first initial cycle, second initial cycle, third initial, fourth initial cycle, and fifth initial cycle. The electronic device 18 comprises an user interface 47 (such as the interface 303 of FIG. 3). The user interface 47 comprises an events button 42, optionally a connectivity button 44 and a timer 45.

The events button 42 can for example be pressed by a user to input information indicative of an event. An event is for example any occurrence of which the sensor data, e.g., the first initial sensor data, second initial sensor data, etc. may be indicative. In some examples, an event is an action performed by the subject. The user can for example input into the electronic device 18, information associated with an event, e.g., a seizure, experienced by the subject (such as a description of symptoms experienced by the subject) via the events button 42. The electronic device 18 may be configured such that when the connectivity button 44 is pressed, e.g., by a user, a connection status page is displayed to the user. The connection status page may comprise information indicative of the quality of connection of the electrodes of the EEG cap 14. The electronic device 18 is for example configured such that, in accordance with a user input indicative of start of an initial cycle, such as the first initial cycle, the timer 45 begins counting down to zero

FIG. 4A shows the electronic device 18A performing an example initial cycle, such as the first initial cycle. The electronic device 18A comprises the events button 42, the connectivity button 44, the timer 45, a first initial instruction 46, and the user interface 47. As shown in FIG. 4A, the first initial instruction 46 may be provided via the user interface 47 of the electronic device 18A. In some examples, the first initial instruction 46 is provided via text, image, video, and/or sound. In some examples, the electronic device 18 is configured to provide a first initial instruction 46 indicative of a first action, e.g., “eye blinking 5 seconds”, via the user interface 47. In some examples, the first initial instruction 46 is associated with the timer 45. The value of the timer 45 is for example associated with the time stated in the first initial instruction 46, e.g., “5 seconds”.

In some examples, the electronic device 18 is configured to obtain first initial sensor data indicative of electrical activity of the brain of the subject. In some examples, the electronic device 18 is configured to determine whether the first initial sensor data is indicative of the first action. In some examples, the electronic device 18 is configured to, in accordance with a determination that the first initial sensor data is indicative of the first action, update the disturbance model based on the first initial sensor data. In some examples, the electronic device 18 is configured to, in accordance with a determination that the first initial sensor data is indicative of the first action, end the first initial cycle. In some examples, the electronic device 18 is configured to, in accordance with a determination that the first initial sensor data is not indicative of the first action, forgo updating the disturbance model based on the first initial sensor data. In some examples, the electronic device 18 is configured to, in accordance with a determination that the first initial sensor data is not indicative of the first action, repeat the first initial cycle, e.g., repeat the first initial cycle until the electronic device 18 determines that the first initial sensor data is indicative of the first action. The electronic device 18 may be configured to repeat the first initial cycle at least N_1 times, e.g., to ensure that initial sensor data is captured multiple times for the same action, such as to improve update of the disturbance model. N_1 may be one, two, three or more. In other words, the first initial cycle may comprise one or more of repetitions of the first initial cycle.

FIG. 4B shows the electronic device 18B performing an example initial cycle, such as the second initial cycle. The electronic device 18B comprises the events button 42, the connectivity button 44, the timer 45, a second initial instruction 48, and the user interface 47. As shown in FIG. 4A, the second initial instruction 48 may be provided via the user interface 47 of the electronic device 18B. In some examples, the second initial instruction 48 is provided via text, image, video, and/or sound. In some examples, the electronic device 18 is configured to provide a second initial instruction 48 indicative of a second action, e.g., “lateral eye movement 5 seconds”, via the user interface 47. In some examples, the second initial instruction 48 is associated with a timer 45. The value of the timer 45 is for example associated with the time stated in the second initial instruction 48, e.g., “5 seconds”.

In some examples, the electronic device 18 is configured to obtain second initial sensor data indicative of electrical activity of the brain of the subject. In some examples, the electronic device 18 is configured to determine whether the second initial sensor data is indicative of the second action. In some examples, the electronic device 18 is configured to, in accordance with a determination that the second initial sensor data is indicative of the second action, update the disturbance model based on the second initial sensor data. In some examples, the electronic device 18 is configured to, in accordance with a determination that the second initial sensor data is indicative of the second action, end the second initial cycle. In some examples, the electronic device 18 is configured to, in accordance with a determination that the second initial sensor data is not indicative of the second action, forgo updating the disturbance model based on the second initial sensor data. In some examples, the electronic device 18 is configured to, in accordance with a determination that the second initial sensor data is not indicative of the second action, repeat the second initial cycle, e.g., repeat the second initial cycle until the electronic device 18 determines that the second initial sensor data is indicative of the second action. The electronic device 18 may be configured to repeat the second initial cycle at least N_2 times, e.g., to ensure that initial sensor data is captured multiple times for the same action, such as to improve update of the disturbance model. N_2 may be one, two, three or more. In other words, the second initial cycle may comprise one or more of repetitions of the second initial cycle.

FIG. 4C shows the electronic device 18C performing an example initial cycle, such as a third initial cycle. The electronic device 18C comprises the events button 42, the connectivity button 44, the timer 45, a third initial instruction 50, and the user interface 47. As shown in FIG. 4A, the third initial instruction 50 may be provided via the user interface 47 of the electronic device 18C. In some examples, the third initial instruction 50 is provided via text, image, video, and/or sound. In some examples, the electronic device 18 is configured to provide a third initial instruction 50 indicative of a third action, e.g., “eyes closed 5 seconds”, via the user interface 47. In some examples, the third initial instruction 50 is associated with the timer 45. The value of the timer 45 is for example associated with the time stated in the third initial instruction 50, e.g., “5 seconds”.

In some examples, the electronic device 18 is configured to obtain third initial sensor data indicative of electrical activity of the brain of the subject. In some examples, the electronic device 18 is configured to determine whether the third initial sensor data is indicative of the third action. In some examples, the electronic device 18 is configured to, in accordance with a determination that the third initial sensor data is indicative of the third action, update the disturbance model based on the third initial sensor data. In some examples, the electronic device 18 is configured to, in accordance with a determination that the third initial sensor data is indicative of the third action, end the third initial cycle. In some examples, the electronic device 18 is configured to, in accordance with a determination that the third initial sensor data is not indicative of the third action, forgo updating the disturbance model based on the third initial sensor data. In some examples, the electronic device 18 is configured to, in accordance with a determination that the third initial sensor data is not indicative of the third action, repeat the third initial cycle, e.g., repeat the third initial cycle until the electronic device 18 determines that the third initial sensor data is indicative of the third action. The electronic device 18 may be configured to repeat the third initial cycle at least N_3 times, e.g., to ensure that initial sensor data is captured multiple times for the same action, such as to improve update of the disturbance model. N_3 may be one, two, three or more. In other words, the third initial cycle may comprise one or more of repetitions of the third initial cycle.

FIG. 4D shows the electronic device 18D performing an example initial cycle, such as a fourth initial cycle. The electronic device 18D comprises the events button 42, the connectivity button 44, the timer 45, a fourth initial instruction 52, and the user interface 47. As shown in FIG. 4A, the fourth initial instruction 52 may be provided via the user interface 47 of the electronic device 18D. In some examples, the fourth initial instruction 52 is provided via text, image, video, and/or sound. In some examples, the electronic device 18 is configured to provide a fourth initial instruction 52 indicative of a fourth action, e.g., look straight forward keep eyes open, via the user interface 47. In some examples, the fourth initial instruction 52 is associated with the timer 45. The value of the timer 45 is for example associated with the time stated in the fourth initial instruction 52, e.g., “5 seconds”.

In some examples, the electronic device 18 is configured to obtain fourth initial sensor data indicative of electrical activity of the brain of the subject. In some examples, the electronic device 18 is configured to determine whether the fourth initial sensor data is indicative of the fourth action. In some examples, the electronic device 18 is configured to, in accordance with a determination that the fourth initial sensor data is indicative of the fourth action, update the disturbance model based on the fourth initial sensor data. In some examples, the electronic device 18 is configured to, in accordance with a determination that the fourth initial sensor data is indicative of the fourth action, end the fourth initial cycle. In some examples, the electronic device 18 is configured to, in accordance with a determination that the fourth initial sensor data is not indicative of the fourth action, forgo updating the disturbance model based on the fourth initial sensor data. In some examples, the electronic device 18 is configured to, in accordance with a determination that the fourth initial sensor data is not indicative of the fourth action, repeat the fourth initial cycle, e.g., repeat the fourth initial cycle until the electronic device 18 determines that the fourth initial sensor data is indicative of the fourth action. The electronic device 18 may be configured to repeat the fourth initial cycle at least N_4 times, e.g., to ensure that initial sensor data is captured multiple times for the same action, such as to improve update of the disturbance model. N_4 may be one, two, three or more. In other words, the fourth initial cycle may comprise one or more of repetitions of the fourth initial cycle.

FIG. 4E shows the electronic device 18E performing an example initial cycle, such as a fifth initial cycle. The electronic device 18E comprises the events button 42, the connectivity button 44, the timer 45, a fifth initial instruction 54, and the user interface 47. As shown in FIG. 4A, the fifth initial instruction 54 may be provided via the user interface 47 of the electronic device 18E. In some examples, the fifth initial instruction 54 is provided via text, image, video, and/or sound. In some examples, the electronic device 18 is configured to provide a fifth initial instruction 54 indicative of a fifth action, e.g., “jaw clenching 5 seconds”, via the user interface 47. In some examples, the fifth initial instruction 54 is associated with the timer 45. The value of the timer 45 is for example associated with the time stated in the fifth initial instruction 54, e.g., “5 seconds”.

In some examples, the electronic device 18 is configured to obtain fifth initial sensor data indicative of electrical activity of the brain of the subject. In some examples, the electronic device 18 is configured to determine whether the fifth initial sensor data is indicative of the fifth action. In some examples, the electronic device 18 is configured to, in accordance with a determination that the fifth initial sensor data is indicative of the fifth action, update the disturbance model based on the fifth initial sensor data. In some examples, the electronic device 18 is configured to, in accordance with a determination that the fifth initial sensor data is indicative of the fifth action, end the fifth initial cycle. In some examples, the electronic device 18 is configured to, in accordance with a determination that the fifth initial sensor data is not indicative of the fifth action, forgo updating the disturbance model based on the fifth initial sensor data. In some examples, the electronic device 18 is configured to, in accordance with a determination that the fifth initial sensor data is not indicative of the fifth action, repeat the fifth initial cycle, e.g., repeat the fifth initial cycle until the electronic device 18 determines that the fifth initial sensor data is indicative of the fifth action. The electronic device 18 may be configured to repeat the fifth initial cycle at least N_5 times, e.g., to ensure that initial sensor data is captured multiple times for the same action, such as to improve update of the disturbance model. N_5 may be one, two, three or more. In other words, the fifth initial cycle may comprise one or more of repetitions of the fifth initial cycle.

In the example shown in FIGS. 4A-4E, the electronic device 18 may be configured such that if the initial sensor data of one or more of the plurality of initial cycles, e.g., first initial cycle, second initial cycle, third initial cycle, fourth initial cycle, and/or fifth initial cycle, is not indicative of the associated action, e.g., eye blinking for the first initial cycle, then one or more of the plurality of initial cycles may be repeated after all initial cycles have been performed (such as performed successfully and/or unsuccessfully). In other words, the electronic device may be configured to repeat an initial cycle in accordance with a determination that the initial sensor data is not indicative of the associated action or until a determination that the initial sensor data is indicative of the associated action.

Examples of the method for EEG capture and related electronic device according to the disclosure are set out in the following items:

Item 1. An electronic device comprising an interface and one or more processors, wherein the electronic device is configured to perform a first cycle, wherein to perform the first cycle comprises to: obtain first sensor data indicative of electrical activity of a brain of a subject;
determine, using a disturbance model, first disturbance data including a first disturbance parameter based on the first sensor data;
determine whether the first disturbance data satisfies one or more first disturbance criteria; and
in accordance with a determination that a first primary disturbance criterion of the one or more first disturbance criteria is satisfied, provide a first primary instruction via the interface.

Item 2. Electronic device according to item 1, wherein the electronic device is configured to, in accordance with a determination that a first secondary disturbance criterion of the one or more first disturbance criteria is satisfied, provide a first secondary instruction via the interface.

Item 3. Electronic device according to any of items 1-2, wherein the electronic device is configured to, after performing the first cycle, perform a second cycle, wherein to perform the second cycle comprises to:
obtain second sensor data indicative of electrical activity of the brain of the subject;
determine, using the disturbance model, second disturbance data including a second disturbance parameter based on the second sensor data;
determine whether the second disturbance data satisfies one or more second disturbance criteria; and
in accordance with a determination that a second primary disturbance criterion of the one or more second disturbance criteria is satisfied, provide a second primary instruction via the interface.

Item 4. Electronic device according to any of items 1-3, wherein the electronic device is configured to, in accordance with a determination that a second secondary disturbance criterion of the one or more second disturbance criteria is satisfied, provide a second secondary instruction via the interface.

Item 5. Electronic device according to any of items 1-4, wherein the electronic device is configured to perform a first initial cycle, wherein to perform the first initial cycle comprises to:
provide a first initial instruction indicative of a first action via the interface;
obtain first initial sensor data indicative of electrical activity of the brain of the subject;
determine whether the first initial sensor data is indicative of the first action; and
in accordance with a determination that the first initial sensor data is indicative of the first action, update the disturbance model based on the first initial sensor data.

Item 6. Electronic device according to any of items 1-5, wherein the electronic device is configured to perform a second initial cycle, wherein to perform the second initial cycle comprises to:
provide a second initial instruction indicative of a second action via the interface;
obtain second initial sensor data indicative of electrical activity of the brain of the subject;
determine whether the second initial sensor data is indicative of the second action; and
in accordance with a determination that the second initial sensor data is indicative of the second action, update the disturbance model based on the second initial sensor data.

Item 7. Electronic device according to any of items 1-6 wherein the first action is one or more of: eye blink, lateral eye movement, chew, close eyes, and jaw clench.

Item 8. Electronic device according to any of items 1-7, wherein to determine first disturbance data comprises to determine the first disturbance parameter over a first time window.

Item 9. Electronic device according to item 8 wherein the first time window is in the range from 10 seconds to 1 minute.

Item 10. Electronic device according to any of items 1-9 as dependent on item 3, wherein to determine second disturbance data comprises to determine a second disturbance parameter over a second time window.

Item 11. Electronic device according to any of items 1-10, wherein to determine disturbance data comprises to determine a probability vector indicative of the probability of a plurality of artifacts, and wherein the first disturbance parameter is based on the probability vector.

Item 12. Electronic device according to any of items 1-11, wherein the electronic device is configured to:
determine whether the first disturbance data satisfies a first stop criterion; and
in accordance with first disturbance data satisfying the first stop criterion, provide a first closing instruction indicative of the end of the first cycle.

Item 13. Electronic device according to any of items 1-12 as dependent on item 3, wherein the electronic device is configured to:
determine whether the second disturbance data satisfies a second stop criterion; and
in accordance with second disturbance data satisfying the second stop criterion, provide a second closing instruction indicative of the end of the second cycle.

Item 14. Electronic device according to any of items 1-13, wherein to determine, using a disturbance model, first disturbance data including a first disturbance parameter based on the first sensor data comprises to process the first disturbance data using a 30 second sliding window with 1 second step size.

Item 15. Electronic device according to any of items 1-14, wherein the electronic device is configured to sample sensor data at a frequency greater than 100 Hz.

Item 16. Electronic device according to any of items 1-15, wherein the first disturbance data is based on one or more first primary disturbance parameters associated with a primary artifact and one or more first secondary disturbance parameters associated with a secondary artifact.

Item 17. A method, performed by an electronic device comprising an interface and one or more processors, of monitoring electrical activity of a brain, wherein the method comprises performing a first cycle wherein performing the first cycle comprises:
obtaining first sensor data indicative of electrical activity of a brain of a subject;
determining, using a disturbance model, first disturbance data including a first disturbance parameter based on the first sensor data;
determining whether the first disturbance data satisfies one or more first disturbance criteria; and
in accordance with a determination that a first primary disturbance criterion of the one or more first disturbance criteria is satisfied, providing a first primary instruction via the interface.

Item 18. Method according to item 17, wherein the method comprises, in accordance with a determination that a first secondary disturbance criterion of the one or more first disturbance criteria is satisfied, providing a first secondary instruction via the interface.

Item 19. Method according to any of items 17-18, wherein the method comprises, after performing the first cycle, performing a second cycle, wherein performing the second cycle comprises:
obtaining second sensor data indicative of electrical activity of the brain of the subject;
determining, using the disturbance model, second disturbance data including a second disturbance parameter based on the second sensor data;
determining whether the second disturbance data satisfies one or more second disturbance criteria; and
in accordance with a determination that a second primary disturbance criterion of the one or more second disturbance criteria is satisfied, providing a second primary instruction via the interface.

Item 20. Method according to any of items 17-19, wherein the method comprises, in accordance with a determination that a second secondary disturbance criterion of the one or more second disturbance criteria is satisfied, providing a second secondary instruction via the interface.

Item 21. Method according to any of items 17-20, wherein the method comprises performing a first initial cycle, wherein performing the first initial cycle comprises:
providing a first initial instruction indicative of a first action via the interface;
obtaining first initial sensor data indicative of electrical activity of the brain of the subject;
determining whether the first initial sensor data is indicative of the first action; and
in accordance with a determination that the first initial sensor data is indicative of the first action, updating the disturbance model based on the first initial sensor data.

Item 22. Method according to any of items 17-21, wherein the method comprises performing a second initial cycle, wherein performing the second initial cycle comprises:
providing a second initial instruction indicative of a second action via the interface;
obtaining second initial sensor data indicative of electrical activity of the brain of the subject;
determining whether the second initial sensor data is indicative of the second action; and
in accordance with a determination that the second initial sensor data is indicative of the second action, updating the disturbance model based on the second initial sensor data.

Item 23. Method according to any of items 17-22 wherein the first action is one or more of: eye blink, lateral eye movement, chew, close eyes, and jaw clench.

Item Method according to any of items 17-23, wherein determining first disturbance data comprises determining the first disturbance parameter over a first time window.

Item 25. Method according to item 24 wherein the first time window is in the range from 10 seconds to 1 minute.

Item 26. Method according to any of items 17-25 as dependent on item 19, wherein determining second disturbance data comprises determining a second disturbance parameter over a second time window.

Item 27. Method according to any of items 17-26, wherein determining disturbance data comprises determining a probability vector indicative of the probability of a plurality of artifacts, and wherein the first disturbance parameter is based on the probability vector.

Item 28. Method according to any of items 17-27, wherein the method comprises:
determining whether the first disturbance data satisfies a first stop criterion; and
in accordance with first disturbance data satisfying the first stop criterion, providing a first closing instruction indicative of the end of the first cycle.

Item 29. Method according to any of items 17-28 as dependent on item 19, wherein the method comprises:
determining whether the second disturbance data satisfies a second stop criterion; and
in accordance with second disturbance data satisfying the second stop criterion, providing a second closing instruction indicative of the end of the second cycle.

Item 30. Method according to any of items 17-29, wherein determining, using a disturbance model, first disturbance data including a first disturbance parameter based on the first sensor data comprises processing the first disturbance data using a 30 second sliding window with 1 second step size.

Item 31. Method according to any of items 17-30, wherein the method comprises sampling sensor data at a frequency greater than 100 Hz.

Item 32. Method according to any of items 17-31, wherein the first disturbance data is based on one or more first primary disturbance parameters associated with a primary artifact and one or more first secondary disturbance parameters associated with a secondary artifact.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that the figures comprise some circuitries or operations which are illustrated with a solid line and some circuitries or operations which are illustrated with a dashed line. The circuitries or operations which are comprised in a solid line are circuitries or operations which are comprised in the broadest example embodiment. The circuitries or operations which are comprised in a dashed line are example embodiments which may be comprised in, or a part of, or are further circuitries or operations which may be taken in addition to the circuitries or operations of the solid line example embodiments. It should be appreciated that these operations need not be performed in order presented. Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, nodes and systems described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program circuitries may include routines, programs, objects, components, data structures, etc. that perform specified tasks or implement specific abstract data types. Computer-executable instructions, associated data structures, and program circuitries represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly to be regarded in an illustrative rather than restrictive sense. The claimed disclosure is intended to cover all alternatives, modifications, and equivalents.

### LIST OF REFERENCES

10, 300 electronic device
10A smart phone
12 subject
13 electrodes of the EEG cap
14 EEG cap
15 data provided from EEG cap to data collector
16 data collector
17 communication link
18 electronic device
19 communication link
20 Server
21 cloud network
42 events button
44 connectivity button
45 timer
46 first initial instruction
47 user interface
48 second initial instruction
50 third initial instruction
52 fourth initial instruction
54 fifth initial instruction
100 method of monitoring electrical activity of a brain
100A method of monitoring electrical activity of a brain
150 I'th initial cycle
S101 Initialize and/or increment i
S102 Providing a first initial instruction
S103 Sampling sensor data
S104 Obtaining first initial sensor data
S106 Determining whether the first initial sensor data is indicative of the first action
S106A Determining S106A whether required number of initial cycles have been performed
S107 Providing feedback indicative of the i′th and/or first initial action not being completed correctly
S108 Updating the disturbance model
S110 Providing a second initial instruction
S112 Obtaining second initial sensor data
S114 Determining whether the second initial sensor data is indicative of the first action
S115 Providing feedback indicative of the second initial action not being completed correctly
S116 Updating the disturbance model
S118 Obtaining first sensor data
S120 Determining first disturbance data
S120A Determining the first disturbance parameter over a first time window
S120B Processing the first disturbance data
S122 Determining whether the first disturbance data satisfies a first stop criterion
S123 Forgo providing a first closing instruction
S124 Providing a first closing instruction
S125 Determining a probability vector
S126 Determining whether the first disturbance data satisfies one or more first disturbance criteria
S126A Determining whether the first disturbance data satisfies a first primary disturbance criterion
S126B Determining whether the first disturbance data satisfies a first secondary disturbance criterion
S127 Providing a first instruction
S128 Providing a first primary instruction
S129 Determining whether the first cycle is complete
S130 Providing a first secondary instruction
S131 Outputting first sensor data
S132 Obtaining second sensor data
S134 Determining second disturbance data
S134A Determining a second disturbance parameter over a second time window.
S136 Determining whether the second disturbance data satisfies a first stop criterion
S137 Forgo providing a second closing instruction
S138 Providing a second closing instruction
S140 Determining whether the second disturbance data satisfies one or more second disturbance criteria
S140A Determining whether the second disturbance data satisfies a second primary disturbance criterion
S140B Determining whether the second disturbance data satisfies the second secondary disturbance criterion
S142 Providing a second primary instruction
S144 Providing a second secondary instruction
S146 Outputting second sensor data

1. An electronic device comprising an interface and one or more processors, wherein the electronic device is configured to perform a first initial cycle, wherein to perform the first initial cycle comprises to:
provide a first initial instruction indicative of a first action via the interface;
obtain first initial sensor data indicative of electrical activity of the brain of the subject;
determine whether the first initial sensor data is indicative of the first action; and
in accordance with a determination that the first initial sensor data is indicative of the first action, update a disturbance model based on the first initial sensor data, and wherein the electronic device is configured to perform a first cycle, wherein to perform the first cycle comprises to:
obtain first sensor data indicative of electrical activity of a brain of a subject;
determine, using the disturbance model, first disturbance data including a first disturbance parameter based on the first sensor data;
determine whether the first disturbance data satisfies one or more first disturbance criteria; and
in accordance with a determination that a first primary disturbance criterion of the one or more first disturbance criteria is satisfied, provide a first primary instruction via the interface.

2. Electronic device according to claim 1, wherein the electronic device is configured to, in accordance with a determination that a first secondary disturbance criterion of the one or more first disturbance criteria is satisfied, provide a first secondary instruction via the interface.

3. Electronic device according to claim 1, wherein the electronic device is configured to, after performing the first cycle, perform a second cycle, wherein to perform the second cycle comprises to:
obtain second sensor data indicative of electrical activity of the brain of the subject;
determine, using the disturbance model, second disturbance data including a second disturbance parameter based on the second sensor data;
determine whether the second disturbance data satisfies one or more second disturbance criteria; and
in accordance with a determination that a second primary disturbance criterion of the one or more second disturbance criteria is satisfied, provide a second primary instruction via the interface.

4. Electronic device according to claim 1, wherein the electronic device is configured to, in accordance with a determination that a second secondary disturbance criterion of the one or more second disturbance criteria is satisfied, provide a second secondary instruction via the interface.

5. Electronic device according to claim 1, wherein the electronic device is configured to perform a second initial cycle, wherein to perform the second initial cycle comprises to:
provide a second initial instruction indicative of a second action via the interface;
obtain second initial sensor data indicative of electrical activity of the brain of the subject;
determine whether the second initial sensor data is indicative of the second action; and
in accordance with a determination that the second initial sensor data is indicative of the second action, update the disturbance model based on the second initial sensor data.

6. Electronic device according to claim 1, wherein the first action is one or more of: eye blink, lateral eye movement, chew, close eyes, and jaw clench.

7. Electronic device according to claim 1, wherein to determine first disturbance data comprises to determine the first disturbance parameter over a first time window.

8. Electronic device according to claim 3, wherein to determine second disturbance data comprises to determine a second disturbance parameter over a second time window.

9. Electronic device according to claim 1, wherein to determine disturbance data comprises to determine a probability vector indicative of the probability of a plurality of artifacts, and wherein the first disturbance parameter is based on the probability vector.

10. Electronic device according to claim 1, wherein the electronic device is configured to:
determine whether the first disturbance data satisfies a first stop criterion; and
in accordance with first disturbance data satisfying the first stop criterion, provide a first closing instruction indicative of the end of the first cycle.

11. Electronic device according to claim 3, wherein the electronic device is configured to:
determine whether the second disturbance data satisfies a second stop criterion; and
in accordance with second disturbance data satisfying the second stop criterion, provide a second closing instruction indicative of the end of the second cycle.

12. Electronic device according to claim 1, wherein to determine, using the disturbance model, first disturbance data including a first disturbance parameter based on the first sensor data comprises to process the first disturbance data using a 30 second sliding window with 1 second step size.

13. Electronic device according to claim 1, wherein the first disturbance data is based on one or more first primary disturbance parameters associated with a primary artifact and one or more first secondary disturbance parameters associated with a secondary artifact.

14. A method, performed by an electronic device comprising an interface and one or more processors, of monitoring electrical activity of a brain, wherein the method comprises performing a first initial cycle, wherein performing the first initial cycle comprises:
providing a first initial instruction indicative of a first action via the interface;
obtaining first initial sensor data indicative of electrical activity of the brain of the subject;
determining whether the first initial sensor data is indicative of the first action; and
in accordance with a determination that the first initial sensor data is indicative of the first action, updating the disturbance model based on the first initial sensor data, and wherein the method comprises performing a first cycle, wherein performing the first cycle comprises:
obtaining first sensor data indicative of electrical activity of a brain of a subject;
determining, using the disturbance model, first disturbance data including a first disturbance parameter based on the first sensor data;
determining whether the first disturbance data satisfies one or more first disturbance criteria; and
in accordance with a determination that a first primary disturbance criterion of the one or more first disturbance criteria is satisfied, providing a first primary instruction via the interface.
